(19) United States
(12) Patent Application Publication
(10) Pub. No.: US 2013/0071936 A1
(43) Pub. Date: Mar. 21, 2013

(54) FRET-BASED ZINC(II) ION INDICATOR

(71) Applicant: The Florida State University Research Foundation,, Tallahassee, FL (US)

(72) Inventors: Lei Zhu, Tallahassee, FL (US); Kesavapillai Sreenath, Tallahassee, FL (US)

(73) Assignee: The Florida State University Research Foundation, Inc., Tallahassee, FL (US)

(21) Appl. No.: 13/621,316

(22) Filed: Sep. 17, 2012

Related U.S. Application Data
(60) Provisional application No. 61/537,141, filed on Sep. 21, 2011.

(51) Int. Cl. C07D 471/06; C07F 9/6561; C07F 9/6558; G01N 21/64

(52) U.S. Cl. 436/81; 546/66; 546/23; 546/22; 546/13

(57) ABSTRACT

The present invention is directed to an indicator for targeting zinc(II) ions in a composition, e.g., a biological sample such as for targeting mitochondrial zinc (II) ions. The present invention is directed to a method for preparing an indicator that targets mitochondrial zinc(II), and a method of measuring the concentration of mitochondrial zinc(II) ions.

### CROSS REFERENCE TO RELATED APPLICATIONS

This application claims priority from U.S. Provisional Application Ser. No. 61/537,141, filed on Sep. 21, 2011, the disclosure of which is incorporated herein as if set forth in its entirety.

### STATEMENT REGARDING FEDERALLY SPONSORED RESEARCH & DEVELOPMENT

This invention was made with Government support under Grant CHE-0809201 awarded by the National Science Foundation and Grant R01GM081382 awarded by the National Institutes of Health. The Government has certain rights in this invention.

### FIELD OF THE INVENTION

The field of the invention relates generally to compounds and methods for the detection of zinc(II) ions in compositions such as biological samples, and more specifically the compounds and methods for the detection of zinc(II) ions in mitochondria.

### BACKGROUND OF THE INVENTION

The proper functions of zinc(II)-dependent biomolecules are sensitively affected by the availability of zinc(II). See S. J. Lippard and J. M. Berg, Principles of Bioinorganic Chemistry, University Science Books, Mill Valley, Calif., 1994; B. L. Vallee and K. H. Falchuk, Physiol. Rev., 1993, 73, 79; J. M. Berg and Y. Shi, Science, 1996, 271, 1081; and W. N. Lipscomb and N. Sträter, Chem. Rev., 1996, 96, 2375. Therefore, homeostasis of zinc(II)— the ability to adjust zinc(II) distribution and to mediate zinc(II) transport on demand—is critical in maintaining the well-being of organisms. See W. Maret, BioMetals, 2001, 14, 187. The disruption of zinc(II) homeostasis leads to diseases, ranging from developmental and immunological disorders to neurodegenerative diseases. See J. Nutr., 2000, 130, Supplement of the issue and C. J. Frederickson, J.-Y. Koh and A. I. Bush, Nat. Rev. Neurosci., 2005, 6, 449.

Motivated by the challenge of determining the distribution and dynamics of zinc(II) in living specimens, significant efforts have been put forth to quantify and to image free, or mobile zinc(II) in vivo and in vitro. See A. Kr [symbol] żel and W. Maret, J. Biol. Inorg. Chem., 2006, 11, 1049; E. L. Que, D. W. Domaille and C. J. Chang, Chem. Rev., 2008, 108, 1517; R. McRae, P. Bagchi, S. Sumalekshmy and C. J. Fahrni, Chem. Rev., 2009, 109, 4780; E. Tomat and S. J. Lippard, Curr. Opin. Chem. Biol., 2010, 14, 225. Among the non-invasive techniques, fluorescence-based methods have received considerable attention due to its adequately high spatial and temporal resolutions and the rapidly growing sophistication and accessibility of fluorescence microscopes. See E. M. Nolan and S. J. Lippard, Acc. Chem. Res., 2009, 42, 193 and Z. Xu, J. Yoon and D. R. Spring, Chem. Soc. Rev., 2010, 39, 1996. To complement fluorescence microscopy, fluorescent indicators for zinc(II) are created based on innovative applications of metal coordination-dependent excited state relaxation processes of fluorophores. See A. P. de Silva, H. Q. N. Gunaratne, T. Gunnlaugsson, A. J. M. Huxley, C. P. McCoy, J. T. Rademacher and T. E. Rice, Chem. Rev., 1997, 97, 1515. Various excited state occurrences, such as internal charge transfer, photo-induced electron transfer, excited state proton transfer, and fluorescence resonance energy transfer (FRET), have been integrated into fluorescent indicators targeting zinc(II). See C. J. Chang and S. J. Lippard, Met. Ions Life Sci., 2006, 1, 321.

Additional challenges in the area of zinc(II) indicator development include the following two issues:

(1) High spatial resolution is one of the most distinctive advantages of fluorescence microscopy over other imaging methods. See R. McRae, P. Bagchi, S. Sumalekshmy and C. J. Fahrni, Chem. Rev., 2009, 109, 4780 and M. Fernández-Suárez and A. Y. Ting, Nat. Rev. Mol. Cell. Biol., 2008, 9, 929. Therefore, fluorescent indicators with defined subcellular localization properties would maximize the impact of fluorescence microscopy in imaging applications. Thus far, only a few synthetic indicators are reported to target organellar zinc(II). See E. Tomat, E. M. Nolan, J. Jaworski and S. J. Lippard, J. Am. Chem. Soc., 2008, 130, 15776 and G. Masanta, C. S. Lim, H. J. Kim, J. H. Han, H. M. Kim and B. R. Cho, J. Am. Chem. Soc., 2011, 133, 5698. Genetically encoded indicators for organellar zinc(II) based on a similar strategy for engineering calcium(II) sensor Cameleon have also appeared. See P. J. Dittmer, J. G. Miranda, J. A. Gorski and A. E. Palmer, J. Biol. Chem., 2009, 284, 16289; J. L. Vinkenborg, T. J. Nicolson, E. A. Bellomo, M. S. Koay, G. A. Rutter and M. Merkx, Nat. Methods, 2009, 6, 737; and Y. Qin, P. J. Dittmer, J. G. Park, K. B. Jansen and A. E. Palmer, Proc. Nat. Acad. Sci. USA, 2011, 108, 7351.

(2) Fluorescent indicators with red emission (λₑₘ close to or >600 nm) are desirable partly because their spectral windows lie outside the range of autofluorescence of biological specimens. The development of red-emitting indicators for zinc(II) has been based on the functionalization of a fluorophore structure with a zinc(II) coordinating ligand. See S. L. Sensi, D. Ton-That, J. H. Weiss, A. Rothe and K. R. Gee, Cell Calcium, 2003, 34, 281; K. Kiyose, H. Kojima, Y. Urano and T. Nagano, J. Am. Chem. Soc., 2006, 128, 6548; B. Tang, H. Huang, K. Xu, L. Tong, G. Yang, X. Liu and L. An, Chem. Commun., 2006, 3609-3611; S. Atilgan, T. Ozdemir and E. U. Akkaya, Org. Lett., 2008, 10, 4065; L. Xue, C. Liu and H. Jiang, Chem. Commun., 2009, 1061; P. Du and S. J. Lippard, Inorg. Chem., 2010, 49, 10753; and X. Lu, W. Zhu, Y. Xie, X. Li, Y. Gao, F. Li and H. Tian, Chem. Eur. J., 2010, 16, 8355. This practice has largely been a trial-and-error process in which the effects of functionalization on the photophysical properties of the fluorophore are hardly predictable.

### SUMMARY OF THE INVENTION

The present invention is directed to a design strategy of developing red-color emitting indicators for targeting mitochondrial zinc(II) ions, to a method for preparing an indicator that targets mitochondrial zinc(II), and to FRET-based indicators. The present invention further provides a method for detecting zinc(II) ions in a composition, such as a biological sample, e.g., mitochondria.

The present invention is directed to a compound capable of measuring the concentration of zinc(II) ion in a composition, such as a biological sample. The compound comprises a covalently-linked assembly of a fluorescence resonance energy transfer donor chromophore and a fluorescence resonance energy transfer acceptor chromophore. The fluorescence resonance energy transfer donor chromophore comprises a conjugated functional group capable of chelating zinc(II) ion.

The present invention is further directed to a compound having the structure:

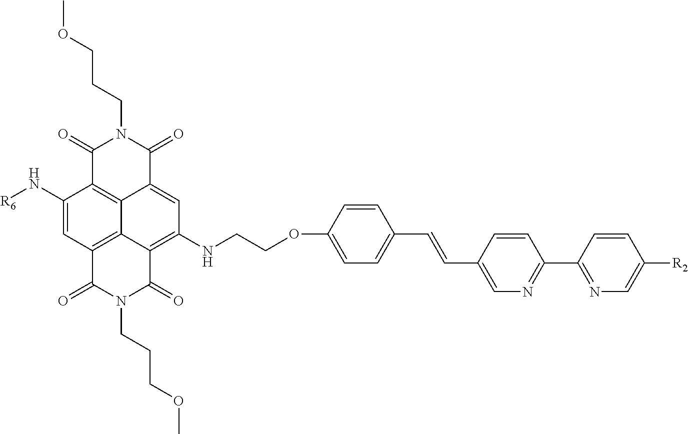

wherein:

R₆ is CH₂—CH₂—CH₂—O—CH₃ or CH₂—CH₂—CH₂—CH₂—CH₂—PPh₃I; and

R₂ is CH₂—O—CH₂—CH₂—O—CH₂—CH₂—O—CH₃ or CH₂—O—CH₂—CH₂-β-CH₂—CH₂—O—CH₃.

The present invention is further directed to a compound having the structure:

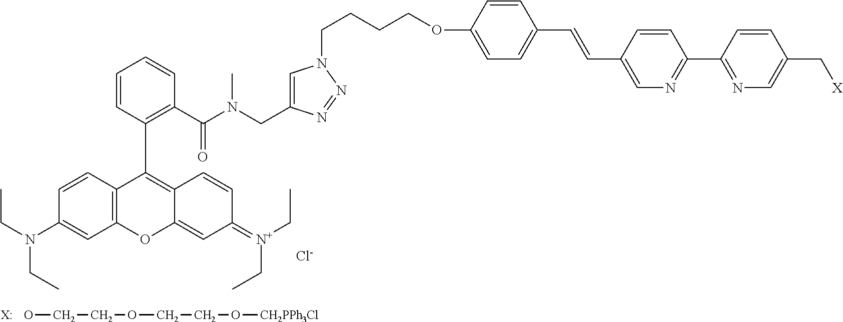

The present invention is further directed to a compound having the structure:

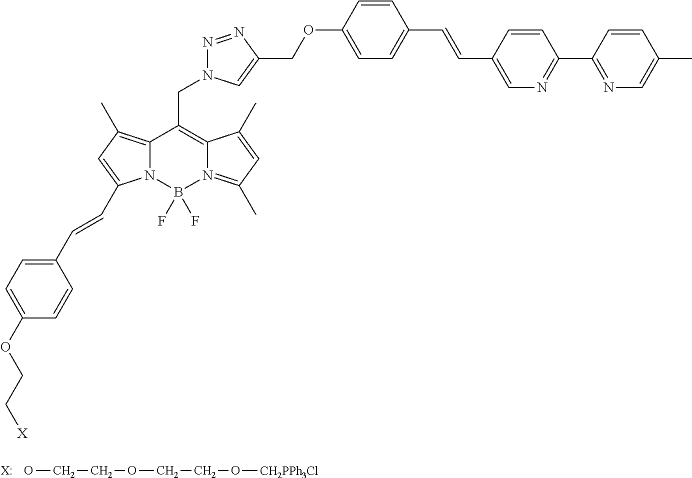

The present invention is further directed to a method of determining the zinc(II) ion concentration in a composition comprising zinc(II) ions. The method comprises contacting the composition comprising zinc(II) ions with a compound comprising a covalently-linked assembly of a fluorescence resonance energy transfer donor chromophore and a fluorescence resonance energy transfer acceptor chromophore. The fluorescence resonance energy transfer donor chromophore comprises a functional group capable of chelating zinc(II) ion, and wherein said contact causes the fluorescence resonance energy transfer donor chromophore to chelate zinc(II) ion. The method further comprises irradiating the composition to thereby excite the zinc(II) ion chelated fluorescence resonance energy transfer donor chromophore, which subsequently transfers the excitation energy (non-radiatively) to the fluorescence resonance energy transfer acceptor chromophore, and allows for the occurrence of emission from the acceptor chromophore. The method further comprises detecting fluorescent emission from the fluorescence resonance energy transfer acceptor chromophore.

### BRIEF DESCRIPTION OF THE DRAWINGS

FIG. 1 is an illustration depicting a strategy for transforming a red fluorophore (rectangle) into a zinc(II) indicator via zinc(II)-induced FRET.

FIG. 2 is a normalized emission spectra of FRET donor compound (1) (left hand peak), zinc-chelated FRET donor compound (1) [Zn(1)]²⁺ (central peak), and absorption spectrum of FRET acceptor compound (2) (right hand peak). The shaded area represent the spectral overlap between the emission of zinc-chelated FRET donor compound (1) [Zn(1)]²⁺ and absorption of FRET acceptor compound (2).

FIG. 3 is a graph depicting fluorescence intensity increase (F/F₀ at 630 nm) of zinc(II) ion indicator (4) (3.0 μM), with increasing [ZnCl₂]. The solid line is the fitting curve of F/F₀ vs. [Zn²⁺] based on a 1:1 binding isotherm equation. See Zhu, L.; Zhong, Z.; Anslyn, E. V. J. Am. Chem. Soc. 2005, 127, 4260-4269. Dissociation constant K_{d}=9.1 μM.

FIGS. 4A and 4B are graphs depicting (4A) Absorption spectra for the titration of zinc(II) ion indicator (4) (3.0 μM) with ZnCl₂ (0-18 μM) in CH₃CN, and (4B) Fluorescence spectral change of zinc(II) ion indicator (4) (3.0 μM) with different concentrations of ZnCl₂ (0-18 μM) in CH₃CN (λₑₓ 400 nm).

FIG. 5 is a graph depicting fluorescence spectral change of zinc(II) ion indicator (4) (2.5 μM) with different concentrations of ZnCl₂ (0-16 μM) in CH₃CN (λₑₓ=600 nm). Inset shows variation of fluorescence intensity at 630 nm as a function of [Zn²⁺].

FIG. 6 is a graph depicting the ratio of emission intensity at 630 nm on excitation of zinc(II) ion indicator (4) (3.0 μM) at 400 and 600 nm vs. [ZnCl₂] in CH₃CN.

FIG. 7 is a graph depicting the effect of addition of ZnCl₂ (0-10 μl) on emission spectrum of an equimolar mixture of FRET donor compound (1) (2.5 μM) and FRET acceptor compound (2) (2.5 μM) in CH₃CN. λₑₓ=400 nm.

FIG. 8 is a chart depicting fluorescence spectroscopic responses of zinc(II) ion indicator (4) (3.0 μM, λₑₓ 400 nm) to various metal ions in 1:9 water/CH₃CN mixture at pH 7.2 (30 mM of MOPS buffer). Clear bars represent intensity in the presence of various metal ions (perchlorate salt). Metal ion concentrations of Na⁺, K⁺, Ca²⁺, Mg²⁺ were 1.0 mM and for other ions 30 μM. Hatch marked bars represent the fluorescence following subsequent addition of Zn(ClO₄)₂ (30 μM).

FIG. 9 is a graph depicting emission spectra for the titration of zinc(II) ion indicator (4) (2.5 μM) with ZnCl₂ (0-22 μM) in water-CH₃CN (1:9) mixture (MOPES buffer, 10 mM, pH=7.2, λₑₓ=400 nm). Inset shows variation of absorbance at 640 nm as a function of [Zn²⁺].

FIG. 10 is a graph depicting the effect of pH on emission spectra of zinc(II) ion chelated FRET donor compound (1) (pH=2.7-10.4). Inset shows variation of fluorescence intensity at 460 nm vs. pH (λₑₓ=380 nm).

FIG. 11 is a graph depicting absorption spectra for the titration of zinc(II) ion indicator (5) (2.4 μM) with Zn(ClO₄)₂ (0-7.4 μM) in CH₃CN.

FIG. 12 is a graph depicting Fluorescence spectral change of zinc(II) ion indicator (5) (2.4 μM) with different concentrations of Zn(ClO₄)₂ (0-14 μM) in CH₃CN (λₑₓ=400 nm).

FIGS. 13A through 13F are confocal fluorescence images of living HeLa (S3) cells transfected with mCerulean3 TOMM-20 that were incubated with zinc(II) ion indicator (5) (1.8 μM, A-C) and zinc(II) ion indicator (4) (6.0 μM, D-F) for 30 min. FIGS. 13A (zinc(II) ion indicator (5)) and 13D (zinc(II) ion indicator (4)) depict the green channel, λₑₓ 405 nm, λₑₘ 425-475 nm. FIGS. 13B (zinc(II) ion indicator (5)) and 13E (zinc(II) ion indicator (4)) depict the red channel, λₑₓ 543 nm, λₑₘ 580-680 nm. FIGS. 13C (zinc(II) ion indicator (5)) and 13F (zinc(II) ion indicator (4)) depict the merged image. Scale bar: 10 μm.

FIGS. 14A through 14F are confocal fluorescence images of live HeLa (S3) cells transfected with mCer3 TOMM20 that were incubated with FRET acceptor compound (3) (9.0 μM, A-C) and FRET acceptor compound (2) (52 μM, D-F) for 30 min. FIGS. 14A (FRET acceptor compound (3)) and 14D (FRET acceptor compound (2)) depict the green channel, λₑₓ 405 nm, λₑₘ 425-475 nm. FIGS. 14B (FRET acceptor compound (3)) and 14E (FRET acceptor compound (2)) depict the red channel, λₑₓ 543 nm, λₑₘ 580-680 nm. FIGS. 14C (FRET acceptor compound (3)) and 14E (FRET acceptor compound (2)) depict the merged image. Scale bar: 10 μm.

FIGS. 15A and 15B are fluorescence images of HeLa (S3) cells incubated with compound (5) (1.8 μM). FIG. 15A depicts no zinc(II) added, and FIG. 15B depicts fluorescence in the presence of 50 μM ZnCl₂. A 405 nm diode laser line was used for excitation. λₑₘ 580-680 nm. Scale bar: 10 μm. The DIC images are included in FIGS. 16A and 16B.

FIGS. 16A and 16B are DIC and fluorescence overlay images of HeLa (S3) cells incubated with compound (5) (1.8 μM). FIG. 16A depicts no zinc(II) added, and FIG. 16B depicts emission in the presence of 50 μM ZnCl₂. A 405 nm diode laser line was used for excitation. λₑₘ 580-680 nm. Scale bar: 10 μm. The gray scale fluorescence images are shown in FIG. 15.

### DETAILED DESCRIPTION OF THE INVENTION

The present invention is directed to a strategy to transform a red fluorophore into an indicator capable of measuring zinc(II) ion concentrations in a sample, particularly a biological sample. The indicator may comprise a mitochondrial targeting moiety enables detection of zinc(II) ions at the cellular level, e.g., in organelles such as mitochondria, thereby enabling the determination of mitochondrial zinc(II) concentrations.

The designed molecule consists of a fluorescent resonance energy transfer (FRET) donor-acceptor pair and, optionally, a mitochondrial targeting moiety. Stated another way, the zinc(II) ion indicator comprises a covalently bonded assembly of a FRET donor chromophore and a FRET acceptor chromophore, and optionally a mitochondrial targeting moiety. The components that make up the zinc(II) ion indicator are covalently bonded. The FRET donor chromophore comprises a conjugated functional group capable of chelating zinc(II) ions.

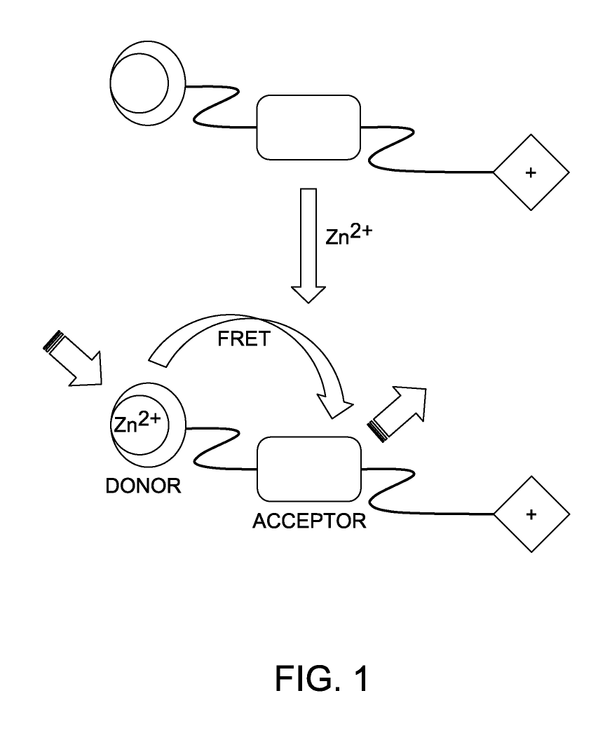

Chelation of zinc(II) ions results in a bathochromic shift in fluorescence emission such that fluorescence emission from the zinc(II) ion chelated FRET donor chromophore substantially overlaps with the absorption spectrum of the FRET acceptor chromophore upon chelation of the FRET donor chromophore with zinc(II) ions. The present invention is therefore further directed to a method in which the zinc(II) ion indicator can target zinc(II) ions in a sample, particularly a biological sample. According to the method of the present invention, the zinc(II) ion indicator is contacted with a sample comprising zinc(II) ions. Said contact enables the indicator to chelate zinc(II) ions. Thereafter, the composition is irradiated such that the zinc(II) ion chelated zinc(II) ion indicator undergoes excitation by light irradiation. Irradiating a composition comprising the zinc(II) ion chelated fluorescence resonance energy transfer donor chromophore causes excitation thereof, which subsequently transfers the excitation energy (non-radiatively) to the fluorescence resonance energy transfer acceptor chromophore, and allows for the occurrence of emission from the acceptor chromophore. The method further comprises detecting fluorescent emission from the fluorescence resonance energy transfer acceptor chromophore. The optional mitochondrial targeting moiety enables detection of zinc(II) ions at the cellular level, e.g., in organelles such as mitochondria. FIG. 1 is an illustration depicting a strategy for transforming a red fluorophore (rectangle) into a zinc(II) indicator via zinc(II)-induced FRET. In FIG. 1, the circles represent free and zinc(II)-bound FRET-donor chromophore. The rectangles represent FRET acceptor chromophore. The incoming and outgoing arrows represent blue excitation and red emission, respectively. The diamond-encompassed “+” on the right represents a lipophilic mitochondrial-targeting cation.

The FRET donor chromophore (represented by the circle in FIG. 1) is a charge-transfer type fluorophore which binds zinc(II) to result in a bathochromic shift of emission. The spectral overlap between donor and acceptor grows upon zinc(II) coordination, thus enhancing the efficiency of FRET. To deliver the probe selectively to biological samples containing zinc(II) ions, such as mitochondria, a lipophilic moiety comprising triphenylphosphonium (TPP) (represented by the diamond-encompassed “+” in FIG. 1), which is a known mitochondrial targeting functionality, is attached through an alkyl chain to the indicator. See M. P. Murphy and R. A. J. Smith, J. Annu. Rev. Pharmacol. Toxicol., 2007, 47, 629. TPP is known to be accumulated in mitochondria. A handful of mitochondrial targeting fluorescent indicators have been constructed using the TPP-tagging strategy, including a case of indicator for zinc(II) by Cho et al. See B. C. Dickinson and C. J. Chang, J. Am. Chem. Soc., 2008, 130, 9638 and G. Masanta, C. S. Lim, H. J. Kim, J. H. Han, H. M. Kim and B. R. Cho, J. Am. Chem. Soc., 2011, 133, 5698.

In some embodiments, the FRET donor chromophore comprises a moiety comprising at least two pyridine functional groups in relatively close proximity such that the pyridine functional groups are capable of chelating zinc(II) ions. The FRET donor chromophore may have the following general structure:

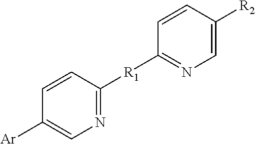

wherein

R₁ is a connecting moiety, preferably a connecting moiety that maintains cooperative coordination (i.e. chelation) between the pyridine functional groups, e.g., a direct covalent bond between the two pyridine functional groups;

R₂ is an alkyl group, a hydroxylalkyl group, an alkoxy group, an alkoxyalkyl group, or a poly(alkoxy) group in which the alkyl moiety may be branched or unbranched, and in which the alkyl moiety may have from 1 to about 12 carbon atoms, from 1 to about 8 carbon atoms, or from 1 to about 4 carbon atoms; and

Ar is an aryl moiety containing a functional group capable of reacting with a functional group on the FRET acceptor chromophore to thereby form the linked assembly of the FRET donor chromophore and the FRET acceptor chromophore.

Preferably, the Ar aryl moiety maintains conjugation with the pyridine functional groups. Stated another way, the Ar moiety may be directly bonded to the pyridine, such that the double bonds or the aryl group and the pyridine are conjugated or the Ar moiety may be linked to the pyridine functional group by conjugated double bonds, e.g., from 1 to about 4. The aryl moiety may comprise one or more ether groups capable of reacting with functional groups on the FRET acceptor chromophore to thereby covalently link the FRET donor chromophore and the FRET acceptor chromophore. Such functional groups include alkoxy, alkoxyalkyl (i.e., ether), or carboxy.

In its native form, an excited state FRET donor chromophore generally emits light within the 380 nm to the 500 nm range. The excitation band of the non-chelated FRET donor centers at 345 nm in acetonitrile, and may cover a range of 300-400 nm. The range of excitation of the zinc(II)-chelated FRET donor is 350-450 nm. Chelation of the FRET donor chromophore generally results in a bathochromic shift (sometimes referred to as a red shift) in the wavelength of excitation/absorption, such as between about 350 nm to about 450 nm, and emission, such as between about 420 to 520 nm. The bathochromic shift causes overlap in the emission spectra of the FRET donor chromophore and the covalently attached FRET acceptor chromophore in the zinc(II) ion indicator. The FRET acceptor chromophore, described more fully below, generally absorbs light in the range of about 500 nm to about 600 nm, and emits fluorescence in the range of about 550 nm to about 700 nm.

In some preferred embodiments, a FRET donor chromophore has the following general structure:

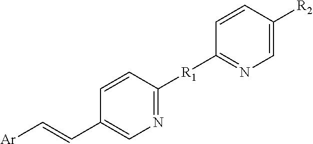

wherein R₁, R₂, and Ar are as defined above.

Exemplary FRET donor chromophores have the following structures:

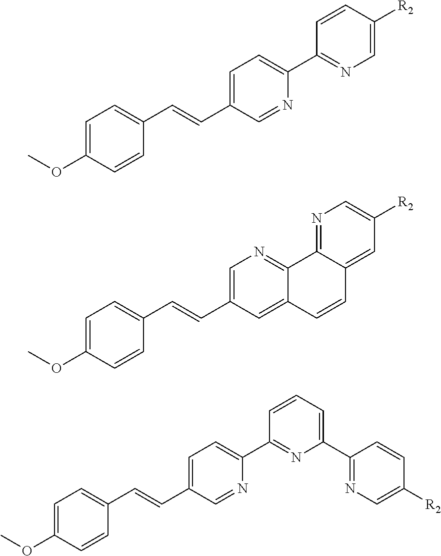

wherein R₁, R₂, and Ar are as defined above.

In preferred embodiments, the R₂ moiety in the above structures is a methyl group. In some embodiments, the R₂ may be modified with a lipophilic moiety comprising triphenylphosphonium (TPP), which is a known mitochondrial targeting functionality. The TPP may be attached through an alkyl chain to the FRET donor chromophore.

In some preferred embodiments, the FRET donor chromophore comprises 5-(4-methoxystyryl)-5′-methyl-2,2′-bipyridine (1) having the following structure:

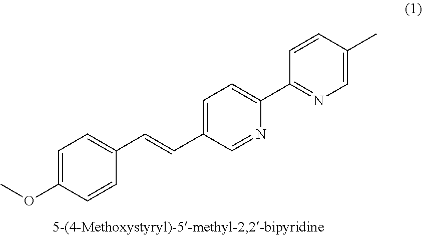

In some embodiments, the FRET acceptor chromophore may be based on naphthalenediimide, a rhodamine (e.g., Rhodamine B), or a boron-dipyrromethene (BODIPY) derivative. In general, the FRET acceptor chromophore absorbs radiation in an overlapping range of wavelengths of the emission of the zinc(II) ion chelated FRET donor chromophore.

In some embodiments, the FRET acceptor chromophore comprises a naphthalenediimide having the following general structure:

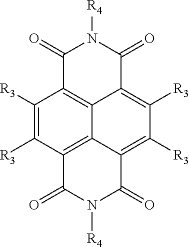

wherein each R₃ and R₄ are independently hydrogen, an alkyl group, a hydroxyalkyl group, an alkoxy group, an alkylalkoxy group, a poly(alkoxy) group, or an alkylamino group. The R₃ and R₄ groups may comprise heteroatoms, particularly oxygen and nitrogen. The R₄ groups may also include aryl (e.g., benzyl or phenyl) or propargyl. Each group may have from 1 to about 12 carbon atoms, which may be branched or unbranched, preferably having from 1 to about 8 carbon atoms, more preferably having from 1 to about 4 carbon atoms. State another way, the alkyl groups of the alkyl, alkoxy moiety, alkylalkoxy, poly(alkylalkoxy), and alkylamino may comprise from 1 to four carbon atoms. In preferred embodiments, two R₃ are alkylamino groups, and in even more preferred embodiments, the two R₃ are on opposite sides of the molecule. In preferred embodiments, at least one of the R₄ group comprises methoxypropyl. In some embodiments, any of the R₃ and R₄ groups may be modified with a functional group capable of bonding to a lipophilic mitochondrial targeting moiety, such as triphenyl-phosphonium. The TPP may be attached through an alkyl chain to the FRET acceptor chromophore.

A napthalenediimide FRET acceptor chromophore may have the following structure:

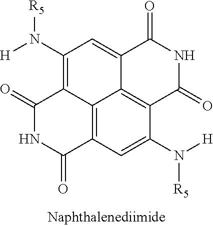

wherein each R₅ is independently hydrogen, an alkyl, a hydroxyalkyl, or an alkoxy. The alkyl or alkoxy may comprise from 1 to about 12 carbon atoms, which may be branched or unbranched, preferably having from 1 to about 8 carbon atoms, more preferably having from 1 to about 4 carbon atoms. In some embodiments, the alkyl or alkoxy comprises a lipophilic mitochondrial targeting moiety, such as triphenyl-phosphonium.

In some preferred embodiments, the FRET acceptor chromophore comprises diamino-substituted naphthalenediimide having the following structures (2) and (3):

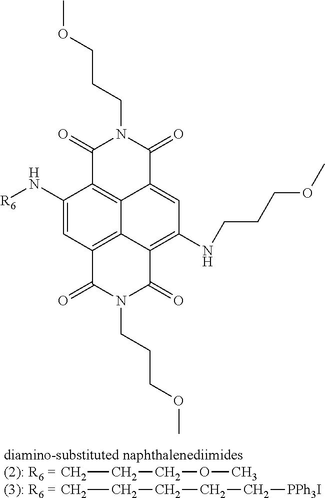

Structure (3) further comprises a lipophilic moiety comprising triphenylphosphonium (TPP), which is a known mitochondrial targeting functionality, attached through an alkyl chain to the FRET acceptor chomophore.

A Rhodamine B FRET acceptor chromophore may have the following structure:

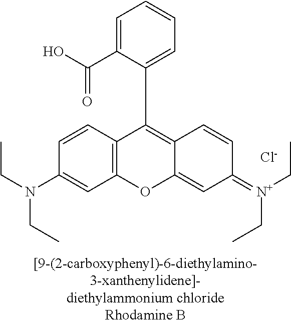 [SMILES: CCN(CC)c1ccc2c(-c3ccccc3C(=O)O)c3ccc(=[N+](CC)CC)cc-3oc2c1 ; [Cl-]]

A boron-dipyrromethene (BODIPY) derivative FRET acceptor chromophore may have the following structure:

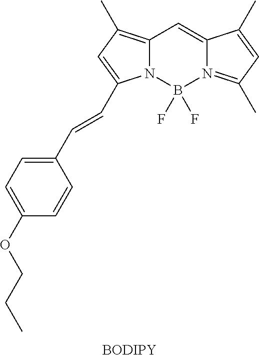

In some preferred embodiments, a FRET donor chromophore and FRET acceptor chromophore may be paired into zinc (II) indicator pairs shown by the following structures (4) and (5):

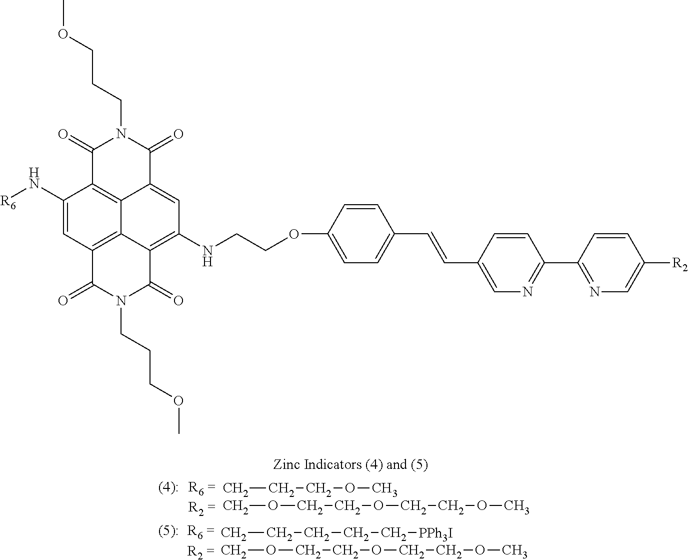

In some preferred embodiments, the zinc(II)-responding indicator based on Rhodamine B as the FRET acceptor chromophore may have the following structure:

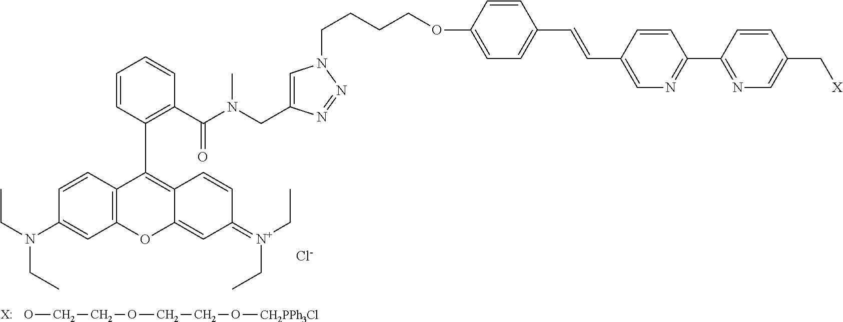

In some preferred embodiments, a zinc(II)-responding indicator based on boron-dipyrromethene (BODIPY) derivative as the FRET acceptor chromophore may have the following structure:

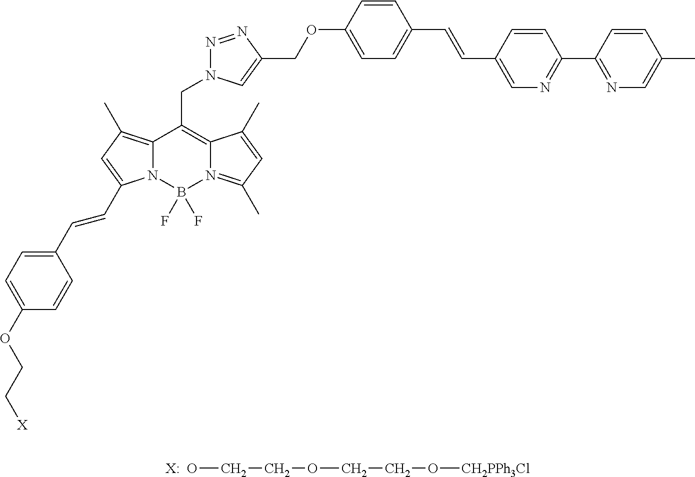

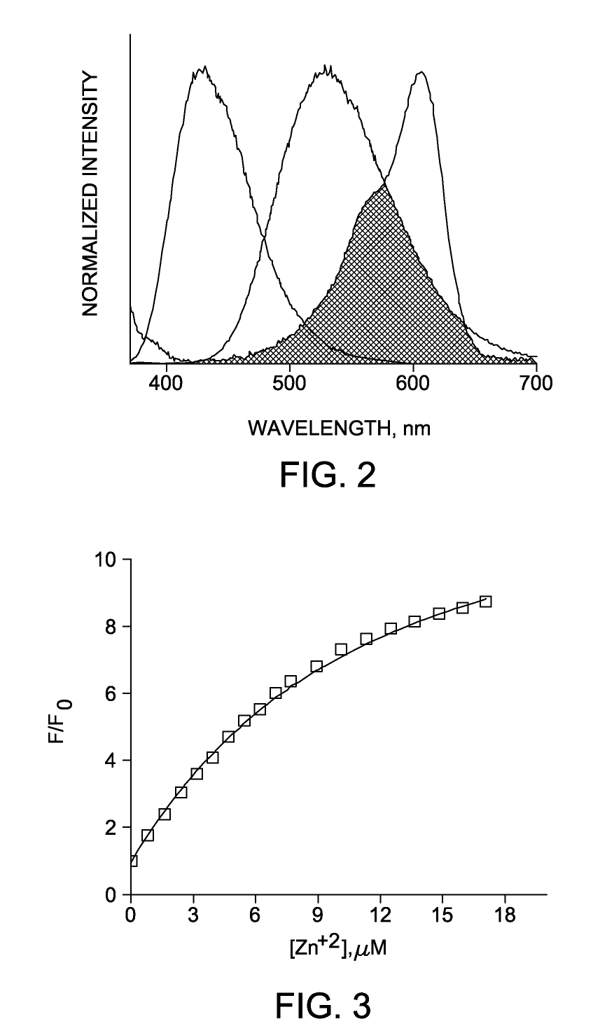

The charge-transferred excited state of FRET donor compound (1) can be stabilized via metal coordination at the bipyridine site. See A. H. Younes, L. Zhang, R. J. Clark and L. Zhu, J. Org. Chem., 2009, 74, 8761 and R. J. Wandell, A. H. Younes and L. Zhu, New J. Chem., 2010, 34, 2176. Consequently, upon addition of zinc(II) to a solution of FRET donor compound (1), its emission spectrum undergoes a bathochromic shift (from blue emission to green emission in FIG. 2) to enable a significant spectral overlap between the emission of zinc(II)-bound (1) and the S₀→S₁ absorption band of FRET acceptor NDI dye (2) (FIG. 2). Naphthalenediimide (NDI) is an advantageous acceptor due to its synthetic versatility, where each of the four sides of NDI can be functionalized individually, and its tunable fluorescence properties. See C. Thalacker, C. Röger and F. Würthner, J. Org. Chem., 2006, 71, 8098. When FRET donor compound (1) and FRET acceptor compound (2) are covalently assembled to afford zinc(II) ion indicator compound (4), the efficiency of intramolecular FRET in zinc(II) ion indicator compound (4) is expected to be a function of zinc(II) concentration. See FIG. 3, which is a graph depicting the increase in normalized fluorescence intensity (F/F₀ at 630 nm) of zinc(II) ion indicator compound (4) (3.0 μM) with increasing [ZnCl₂].

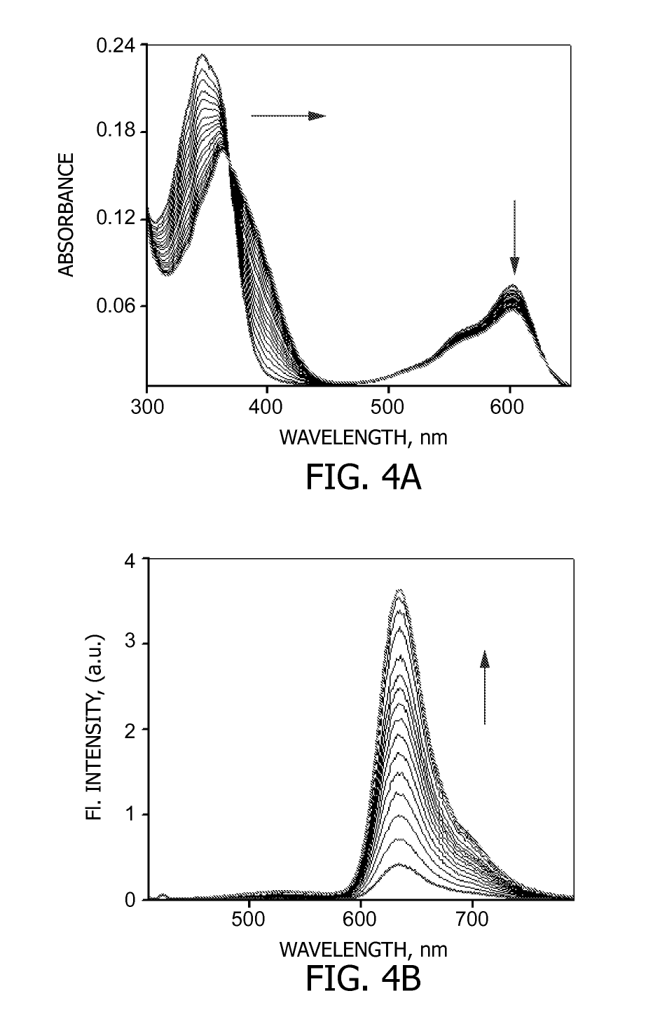

The syntheses of the separate FRET donor and acceptor systems (1-3) and indicators for zinc(II) (4, 5) are described in the examples. Absorption spectrum of zinc(II) ion indicator compound (4) in CH₃CN consists of two bands over 300 nm (FIG. 4A). The band centered at 346 nm corresponds to the absorption of the bipy-containing FRET donor moiety, which we term the D band. The band centered at 600 nm corresponds to the NDI moiety which is termed the A band. Upon addition of increasing amount of ZnCl₂, as a result of zinc(II) coordination at the bipy moiety, the charge-transfer D band undergoes a bathochromic shift from 346 nm to 370 nm. The A band, meanwhile, experiences a slight hypochromic shift.

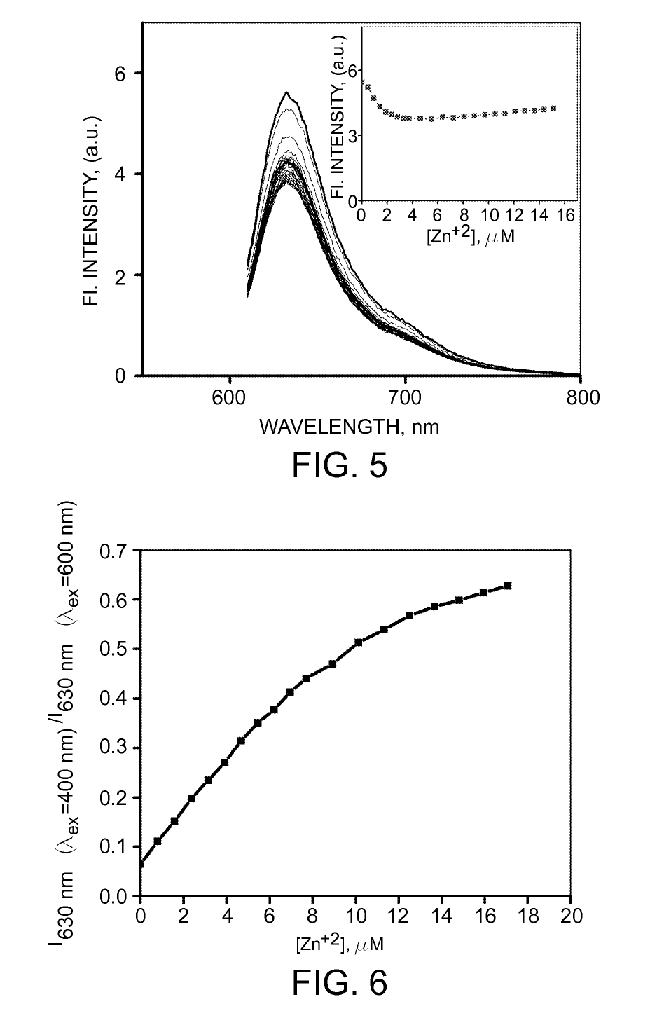

The fluorescence titration profile of zinc(II) ion indicator compound (4) with ZnCl₂ upon excitation at 400 nm is shown in FIG. 4B. The spectrum of zinc(II) ion indicator (4) exhibits a weak emission at 630 nm. This signal may have arisen from the direct excitation of the NDI component. Upon addition of increasing amount of ZnCl₂ from 0-18 μM, the emission at 630 nm intensifies, and reaches saturation with 12-fold enhancement in integrated intensity (See Table 1 for spectroscopic data). However, when NDI is directly excited at 600 nm, the intensity is not affected much by the variation of zinc(II) concentration ([Zn²⁺]) (FIG. 5). These observations are consistent with the involvement of a zinc(II)-modulated intramolecular excitation energy transfer process. Furthermore, the differed sensitivity to excitation wavelength of zinc(II) ion indicator (4) offers an opportunity to correlate [Zn²⁺] and fluorescence change ratiometrically. See P. Carol, S. Sreejith and A. Ajayaghosh, Chem. Asian J., 2007, 2, 338. As shown in FIG. 6, the ratio of integrated fluorescence upon excitation at 400 and 600 nm is sensitively dependent on [Zn²⁺]. This correlation is independent of the concentration of zinc(II) ion indicator compound (4) in the linear portion of the calibration curve.

TABLE 1
Spectroscopic data of 1-5 and ZnCl₂
complexes of (1), (4), and (5) in CH₃CN.

| Compound | λ_{abs} [nm] | ∈/10³[M⁻¹ cm⁻¹] | λₑₘ [nm] | φ_{f} |
|---|---|---|---|---|
| (1) | 345 | 34 | 428 | 0.28^{a} |
| [Zn(1)]^{2+,b} | 374 | 28 | 528 | 0.46^{a} |
| (2) | 607 | 22 | 638 | 0.51^{c} |
| (3) | 607 | 23 | 639 | 0.48^{c} |
| (4) | 346 (D) | 76 | 633 | 0.41^{c} |
| | 600 (A) | 22 | | |
| [Zn(4)]^{2+,d} | 360 (D) | 55 | 633 | 0.31^{e} |
| | 600 (A) | 21 | | |
| (5) | 347 (D) | 81 | 633 | 0.28^{c} |
| | 602 (A) | 23 | | |
| [Zn(5)]^{2+,d} | 363 (D) | 67 | 633 | 0.29^{e} |
| | 602 (A) | 21 | | |

^{[a]} λₑₓ = 365 nm,
^{[b]} data taken from Younes, A. H.; Zhang, L.; Clark, R. J.; Zhu, L. J. Org. Chem. 2009, 74, 8761-8772,
^{[c]} λₑₓ = 565 nm,
^{[d]} Zn (ClO₄) ₂ was added in 10 equiv. relative to 4 or 5,
^{[e]} λₑₓ = 400 nm.

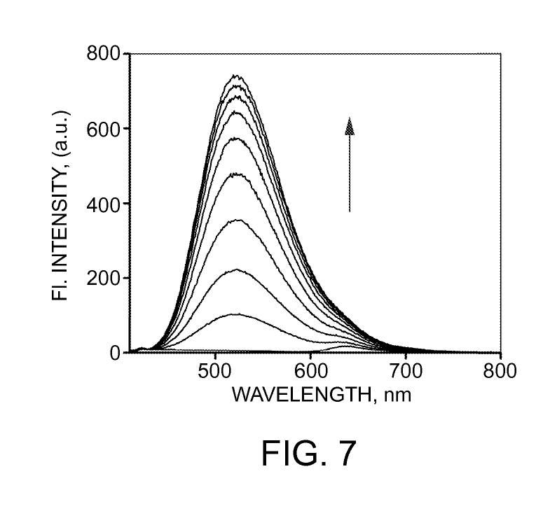

The sensitized excitation of the NDI moiety in zinc(II) ion indicator (4) when excited at 400 nm is supported by the observation of the following control experiment. ZnCl₂ was titrated into a mixture of FRET donor compound (1) and FRET acceptor compound (2) of equal concentration (2.5 μM) while the emission spectra were collected upon excitation at 400 nm. Only a weak band of NDI was seen at the beginning of the titration which was quickly overwhelmed by the growing green emission from [Zn(1)]²⁺ (FIG. 7). In zinc(II) ion indicator (4) however, the excitation energy of the zinc(II)-bound donor portion, which is equivalent to [Zn(1)]²⁺, is captured by the NDI acceptor, which emits instead to results in the observations in FIG. 4B.

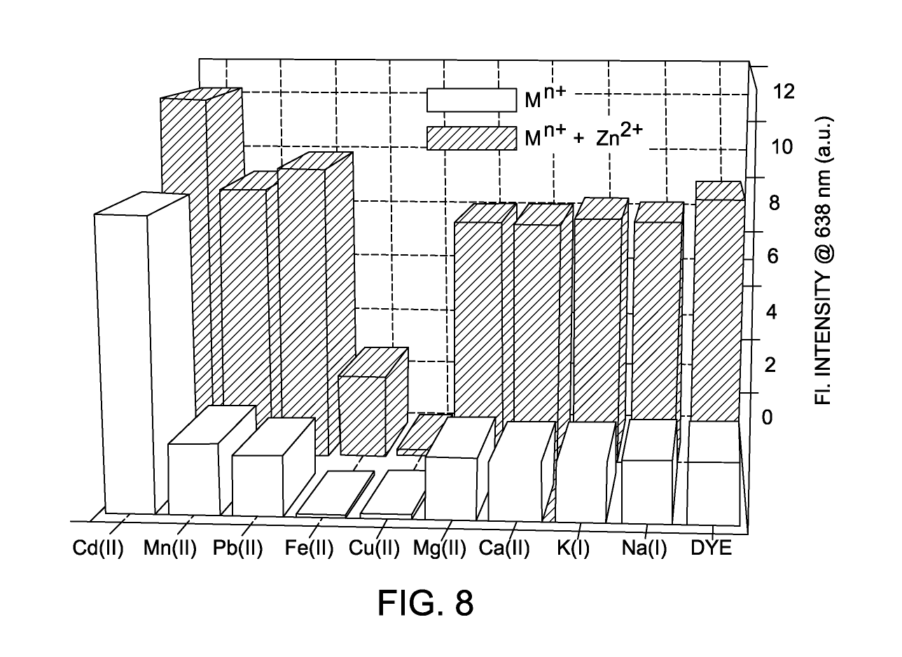

The fluorescence dependence of zinc(II) ion indicator (4) on the identity of metal ion was evaluated (FIG. 8). The alkali and alkaline earth metal ions that are abundant in biological systems do not elicit a fluorescence response of (4). Although manganese(II) and lead(II) are known to bind bipy, they fail to alter the fluorescence of zinc(II) ion indicator (4). Not surprisingly, cadmium(II) acts similarly to zinc(II) in resulting strong fluorescence from NDI, whereas iron(II) and copper(II) quench fluorescence. The observed metal ion selectivity can be understood based on the Irving-Williams series and HSAB theory. Although cadmium(II), iron(II), and copper(II) are potential interfering ions, the applicability of zinc(II) ion indicator (4) in microscopic imaging experiments shall not be significantly compromised due to the low abundances of these ions in biological

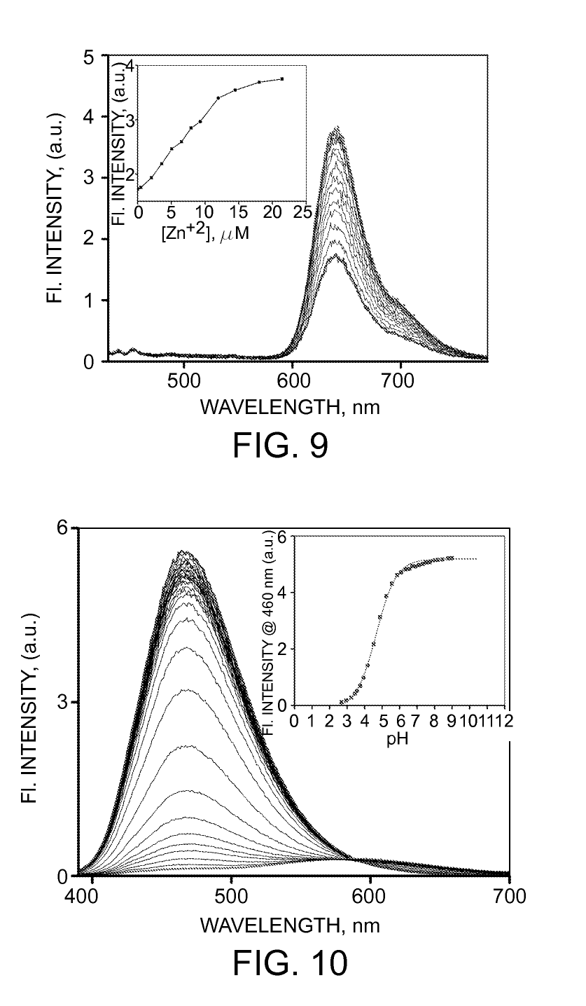

The titration experiments using zinc(II) ion indicator (4) were also carried out in an aqueous/organic mixed solvent (1:9 MOPS buffer (pH 7.2)/CH₃CN). Zinc(II) ion indicator (4) shows a 3-fold fluorescence enhancement on increasing [Zn²⁺] (FIG. 9). Comparing to the observations in CH₃CN, the fluorescence is rather dim. The loss of fluorescence can be explained by the hydrogen bonding interaction between the protic solvent and the carbonyl groups on NDI, which provides a non-radiative deactivation pathway. See F. Würthner, S. Ahmed, C. Thalacker and T. Debaerdemaeker, Chem. Eur. J., 2002, 8, 4742. The pKₐ of FRET donor compound 1, which is also the ionophore of zinc(II) ion indicator compound 4, is 4.6. The intensity of zinc(II) ion chelated FRET donor compound 1 exhibits little change in the range of pH 6.0-9.0 (FIG. 10). By extension, the fluorescence of zinc(II) ion indicators (4) and (5) would not be affected by the fluctuations of pH within the physiological range.

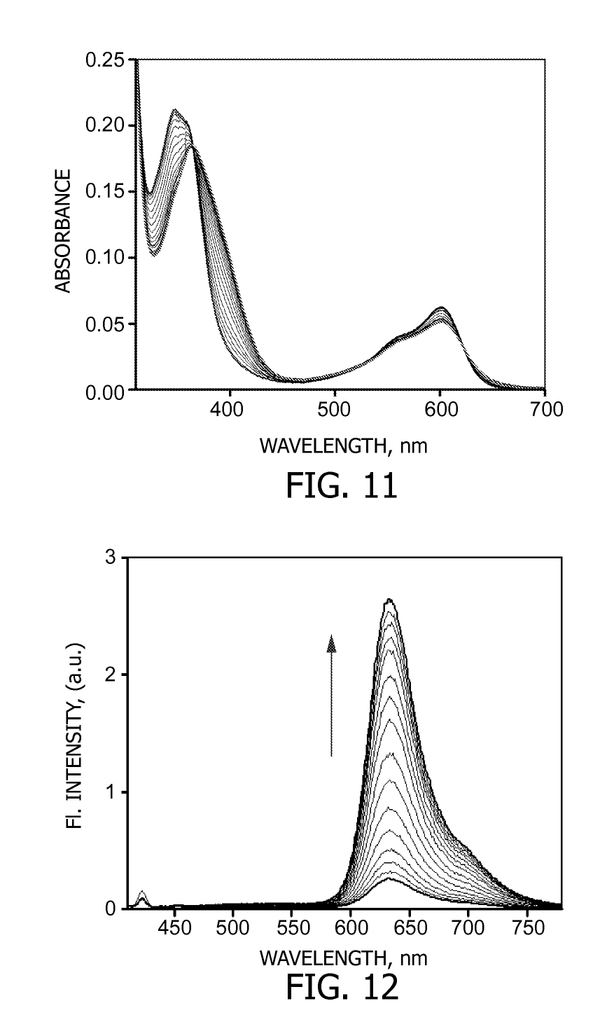

To equip the indicator with mitochondrial targeting capability, the indicator may be modified by covalently bonding a lipophilic mitochondrial targeting moiety. In some embodiments, the targeting moiety comprises a triphenylphosphonium (TPP) group covalently bonded to the FRET acceptor compound (2) and zinc(II) ion indicator (4) to afford FRET acceptor compound (3) and zinc(II) ion indicator (5), respectively. See FIGS. 11 and 12, which are graphs depicting the absorption spectra and fluorescence emission of zinc(II) ion indicator (5) titrated with 0 to 7.4 μM Zn(ClO₄)₂.

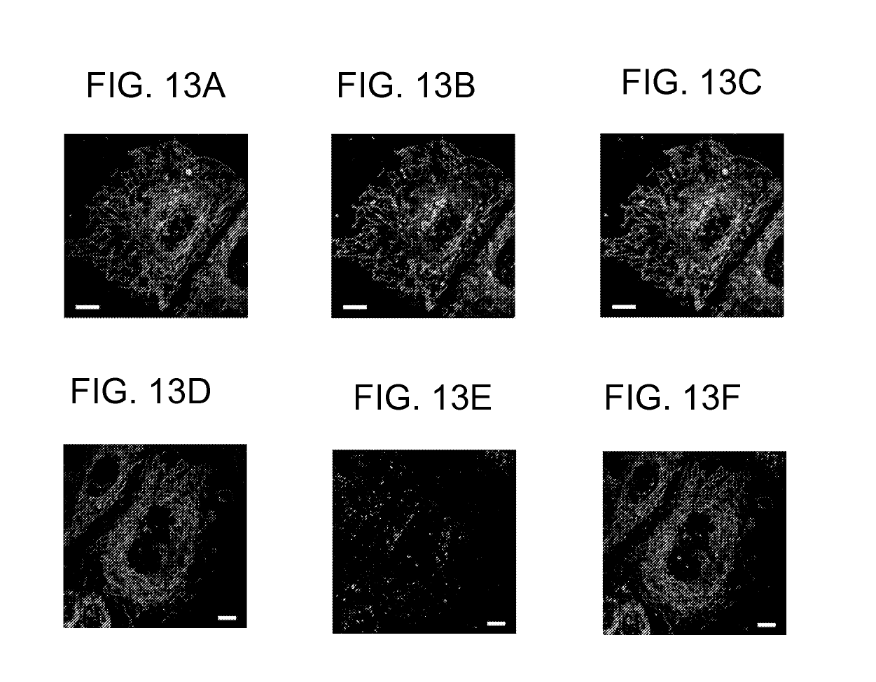

The colocalization properties of red-emitting compounds (2-5) were studied using HeLa (S3) cells transfected with mCerulean3 TOMM-20, a cyan fluorescent protein (CFP) fused with a mitochondrial targeting sequence whose emission is shown in the green channel. See FIG. 13A for emission spectrum from cells incubated with TPP-containing zinc(II) ion indicator (5), and see FIG. 13D for emission spectrum from cells incubated with zinc(II) ion indicator (4). Lacking the TPP group, zinc(II) ion indicator (4) forms particulates intracellularly (FIG. 13E, which depicts emission spectrum from cells incubated with zinc(II) ion indicator (4)) which show poor colocalization with the CFP (FIG. 13F, merged image). The overall intracellular fluorescence intensity from zinc(II) ion indicator (4) is low, which is consistent with the observed weak fluorescence of the NDI fluorophore in aqueous solutions. The TPP-containing zinc(II) ion indicator (5) (FIG. 13B, which depicts emission spectrum from cells incubated with TPP-containing zinc(II) ion indicator (5)), on the other hand, localizes selectively in mitochondria (FIG. 13C) and displays a surprisingly strong intracellular fluorescence relative to that of zinc(II) ion indicator (4).

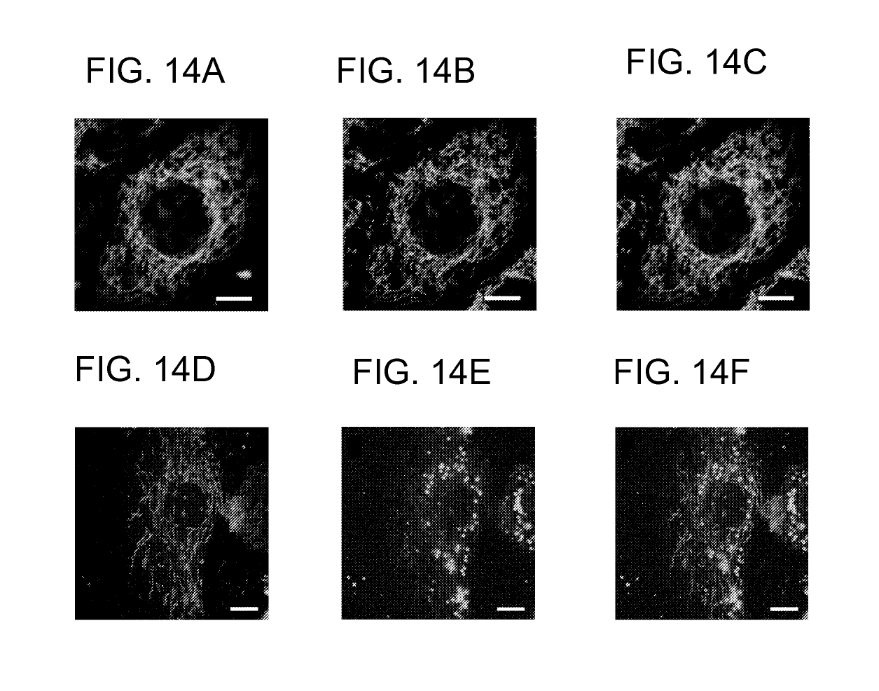

Similar contrast was observed between the model FRET acceptor compounds (2) and (3) (FIGS. 14A through 14F). The strong fluorescence of intracellular zinc(II) ion indicator (5) may be attributed to the dispersion of the indicator molecules into the inner mitochondrial membrane or a relatively organic-like mitochondrial matrix. See M. P. Murphy and R. A. J. Smith, J. Annu. Rev. Pharmacol. Toxicol., 2007, 47, 629.

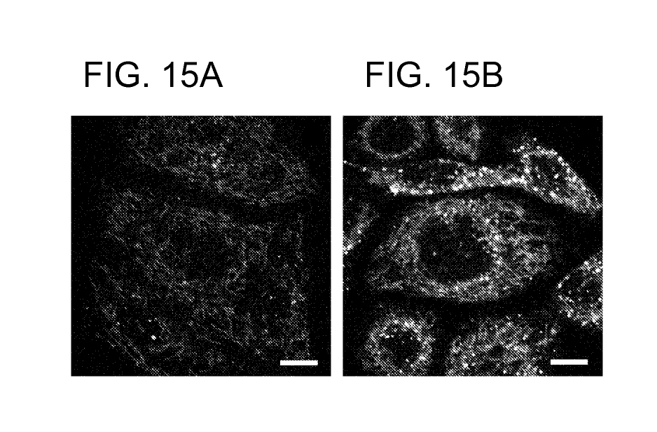

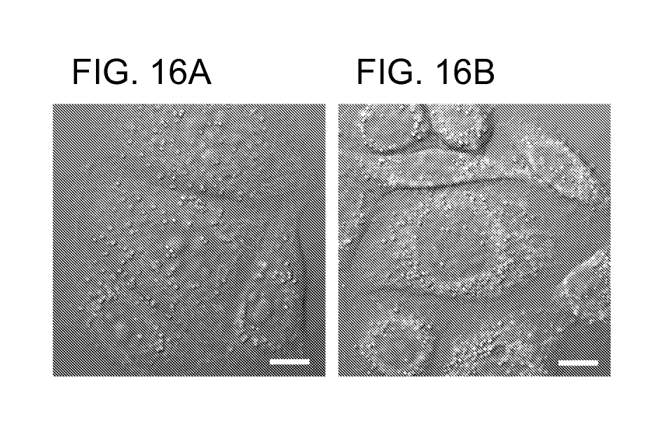

The ability of zinc(II) ion indicator 5 to report mitochondrial zinc(II) variation is demonstrated in FIGS. 15A and 15B. See also FIGS. 16A and 16B. Upon excitation at 405 nm, the addition of supplemental zinc(II) in the growth media (DMEM) leads to an increase in intramolecular fluorescence of zinc(II) ion indicator (5) from the red channel that largely localizes in mitochondria. The addition of zinc(II) ion indicator (5) to the group of very few synthetic indicators that target mitochondrial zinc(II) shall aid the characterization of mitochondrial zinc(II) which is intimately correlated to the chemistry of reactive oxygen species (ROS). See E. Tomat, E. M. Nolan, J. Jaworski and S. J. Lippard, J. Am. Chem. Soc., 2008, 130, 15776; G. Masanta, C. S. Lim, H. J. Kim, J. H. Han, H. M. Kim and B. R. Cho, J. Am. Chem. Soc., 2011, 133, 5698; Y. Qin, P. J. Dittmer, J. G. Park, K. B. Jansen and A. E. Palmer, Proc. Nat. Acad. Sci. USA, 2011, 108, 7351; and S. L. Sensi, D. Ton-That, P. G. Sullivan, E. A. Jonas, K. R. Gee, L. K. Kaczmarek and J. H. Weiss, Proc. Natl. Acad. Sci. U.S.A., 2003, 100, 6157.

In summary, the conversion of a red-emitting fluorophore to a zinc(II)-responding indicator using a FRET-based strategy is demonstrated. The diamino-substituted NDI dye, which has not drawn much attention in intracellular applications, performs well as the fluorophore in indicator 5. See X. Lu, W. Zhu, Y. Xie, X. Li, Y. Gao, F. Li and H. Tian, Chem. Eur. J., 2010, 16, 8355. In addition to red-emitting, zinc(II) ion indicator 5 offers attractive features including a large spectral separation between excitation and emission, little pH sensitivity within the physiological window, ratiometric imaging capability, and defined subcellular localization preference.

### EXAMPLES

The following non-limiting examples are provided to further illustrate the present invention.

### I. Materials and General Methods

Reagents and solvents were purchased from various commercial sources and used without further purification unless otherwise stated. CH₃CN (OmniSolv, EMD) were directly used in titration experiments without purification. All reactions were carried out in oven- or flame-dried glassware. Analytical thin-layer chromatography (TLC) was performed using pre-coated TLC plates with silica gel 60 F254 (EMD) or with aluminum oxide 60 F254 neutral (EMD). Flash column chromatography was performed using 40-63 μm (230-400 mesh ASTM) silica gel (EMD) or alumina (80-200 mesh, pH 9-10, EMD) as the stationary phases. Silica and alumina gel was flame-dried under vacuum to remove absorbed moisture before use. ¹H NMR spectra were recorded at 300 MHz on a Varian Mercury spectrometer. ¹³C NMR spectra were recorded at 125 MHz, on a Bruker Avance spectrometer. All chemical shifts were reported in δ units relative to tetramethylsilane. High resolution mass spectra were obtained at the Mass Spectrometry Laboratory at Florida State University. ESI spectra were obtained on a JEOL AccuTOF spectrometer. Spectrophotometric and fluorometric titrations were conducted on a Varian Cary 100 Bio UV-Visible Spectrophotometer and a Varian Cary Eclipse Fluorescence Spectrophotometer, respectively, with a 1-cm standard quartz cell. The fluorescence quantum yield was determined by comparison of the integrated area of corrected emission spectrum with reference of N,N′-di-n-octyl-2,6-di-n-octylamino-1,4,5,8-naphthalenetetracarboxylic acid diimide (φ_{f}=0.53 in CH₂Cl₂) as the reference by the literature method. See Thalacker, C.; Röger, C.; Würthner, F. J. Org. Chem. 2006, 71, 8098-8105.

### II. Syntheses and Characterizations of New Compounds

### Example 1

### Synthetic Scheme for Compound (2)

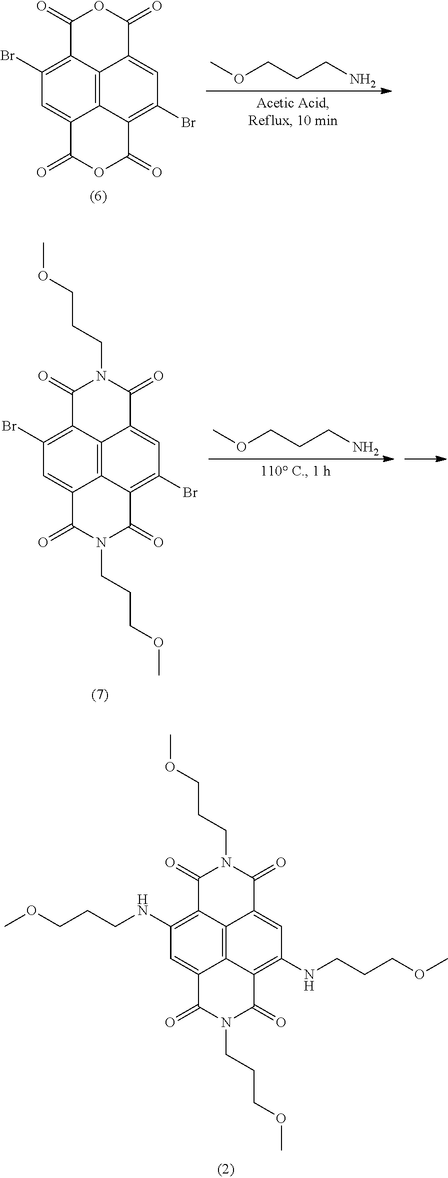

Compound (7).

2,6-Dibromonaphthalene-1,4,5,8-tetracarboxylic acid bisanhydride (6) (400 mg, 0.94 mmol) was suspended in glacial acetic acid (20 mL). See Bell, T. D. M.; Yap, S.; Jani, C. H.; Bhosale, S. V.; Hofkens, J.; De Schryver, F. C.; Langford, S. J.; Ghiggino, K. P. Chem. Asian J. 2009, 4, 1542-1550. To the stirred suspension 3-methoxypropylamine (1.0 mL, 9.7 mmol) was added slowly at room temperature (“rt”) and the mixture was kept at reflux at 120° C. for 10 min. The reaction mixture was cooled to rt and the resulting pale yellow precipitate was collected and washed with glacial acetic acid. The yield of (7) was 198 mg (37%). ¹H NMR (300 MHz, CDCl₃, 25° C.) 8.99 (s, 2H), 4.32 (t, J=7.2 Hz, 4H), 3.52 (t, J=6.0 Hz, 4H), 3.29 (s, 6H), 2.04 (m, 4H).

Compound (2).

Compound (7) (50 mg, 0.09 mmol) and 3-methoxypropylamine (2.0 mL) were refluxed under an argon atmosphere for 1 h. Then the reaction mixture was diluted with CH₂Cl₂ (50 mL) followed by extraction with a dilute HCl solution (0.5 M, saturated with NaCl) three times. The organic layer was dried over anhydrous Na₂SO₄ before the solvent was removed under a reduced pressure and the obtained blue solid was purified by silica chromatography using THF in CH₂Cl₂ (gradient 0-10%). The yield of (2) was 32 mg (63%). ¹H NMR (300 MHz, CDCl₃, 25° C.) 9.40 (t, 2H), 8.10 (s, 2H), 4.26 (t, J=7.2 Hz, 4H), 3.51-3.61 (m, 12H), 3.39 (s, 6H), 3.34 (s, 6H), 1.96-2.07 (m, 8H). ¹³C NMR (125 MHz, CDCl₃, 25° C.) 166.1, 163.1, 149.2, 125.7, 121.1, 118.3, 101.9, 70.8, 70.2, 59.0, 58.8, 40.6, 38.0, 29.6, 28.3. HRMS (ESI+): calcd. (M+Na⁺) 607.2744. found 607.2754.

### Example 2

### Synthetic Scheme for Compound (3)

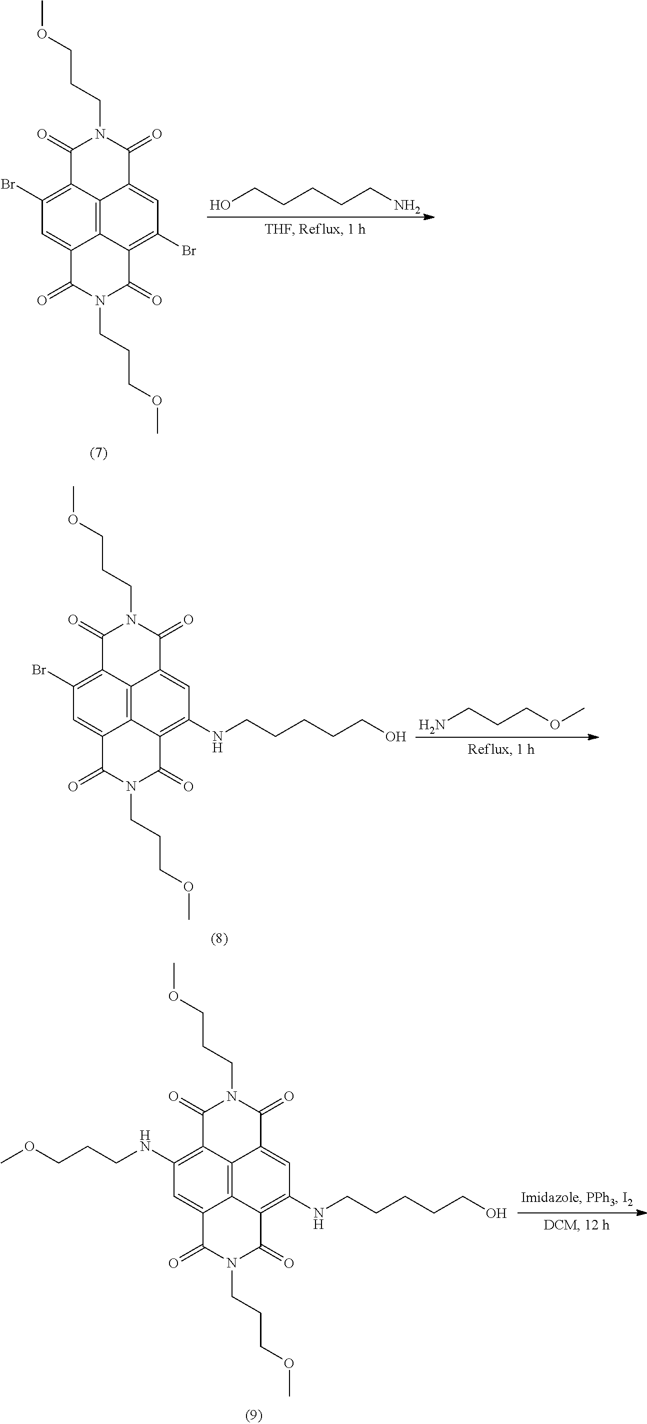

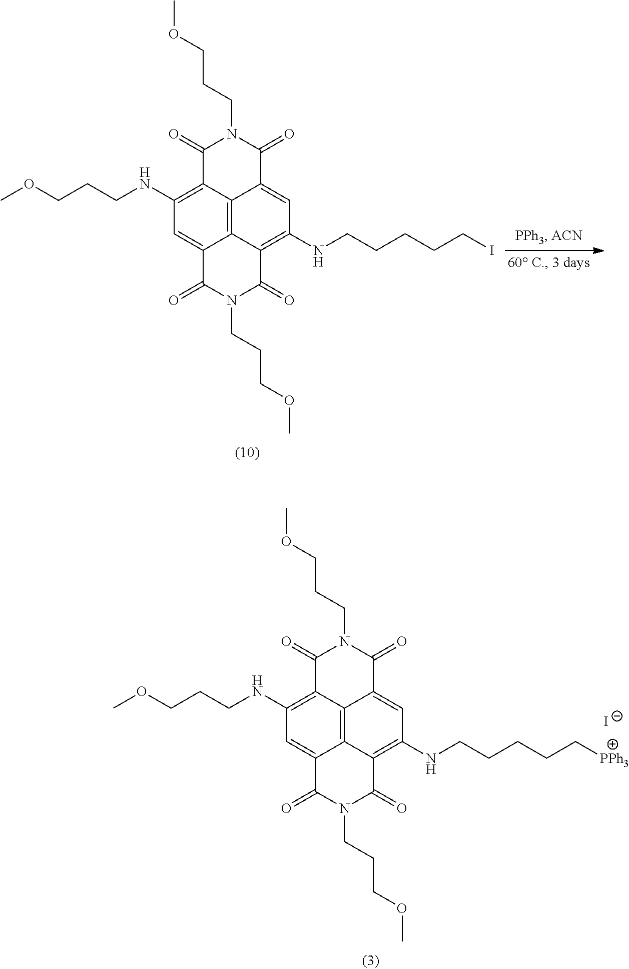

Compound (8).

Compound (7) (50 mg, 0.09 mmol) and 5-aminopentanol (100 mg, 0.97 mmol) were refluxed in THF for 1 h. The solvent was removed under a reduced pressure and the obtained red solid was purified by silica chromatography using THF in CH₂Cl₂ (gradient 0-20%). The yield of 8 was 27 mg (53%). ¹H NMR (300 MHz, CDCl₃, 25° C.) 10.12 (t, 1H), 8.08 (s, 1H), 8.23 (s, 1H), 4.25 (t, 4H), 3.68 (q, 2H), 3.56 (q, 2H), 3.51 (t, 4H), 3.29 (s, 6H), 1.93-2.04 (m, 4H), 1.84 (q, 2H), 1.56-1.68 (m, 4H). MS (ESI−): calcd. 589.4. found 589.4.

Compound (9).

Compound (8) (20 mg, 0.03 mmol) and 3-methoxypropylamine (1.0 mL) were refluxed under an argon atmosphere for 1 h. Then the reaction mixture was diluted with CH₂Cl₂ (50 mL) followed by extraction with a dilute HCl solution (0.5 M, saturated with NaCl) three times. The organic layer was dried over anhydrous Na₂SO₄ before the solvent was removed under a reduced pressure and the obtained blue solid was purified by silica chromatography using THF in CH₂Cl₂ (gradient 0-20%). The yield was 14 mg (70%). ¹H NMR (300 MHz, CDCl₃, 25° C.) 9.41 (t, 1H), 9.34 (t, 1H), 8.11 (s, 1H), 8.08 (s, 1H), 4.26 (t, J=7.2 Hz, 4H), 3.71 (q, 2H), 3.47-3.63 (m, 10H), 3.39 (s, 3H), 3.35 (s, 6H), 1.98-2.06 (m, 6H), 1.84 (q, 2H), 1.53-1.68 (m, 4H). ¹³C NMR (125 MHz, CDCl₃, 25° C.) 166.0, 165.9, 162.9, 162.8, 149.0, 148.9, 125.4, 120.8, 118.0, 101.6, 101.5, 70.6, 70.1, 62.6, 58.8, 58.6, 43.0, 40.4, 37.9, 32.3, 29.5, 29.1, 28.2, 23.4. HRMS (ESI+): calcd. (M+Na⁺) 621.2900. found 621.2919.

Compound (10).

PPh₃ (42 mg, 0.16 mmol) and imidazole (11 mg, 0.16 mmol) were dissolved in dry CH₂Cl₂ (4.0 mL) and cooled to 0° C. Iodine (40 mg, 0.16 mmol) was added and stirred for 30 min. Compound (9) (50 mg, 0.08 mmol) was dissolved in dry CH₂Cl₂ (1.0 mL) was added dropwise and stirred for 12 h at rt. The solvent was removed under a reduced pressure and the obtained blue solid was purified by silica chromatography using THF in CH₂Cl₂ (gradient 0-20%). The yield of (10) was 41 mg (70%). ¹H NMR (300 MHz, CDCl₃, 25° C.) 9.44 (t, 1H), 9.36 (t, 1H), 8.16 (s, 1H), 8.10 (s, 1H), 4.27 (t, J=7.2 Hz, 4H), 3.48-3.64 (m, 10H), 3.39 (s, 3H), 3.35 (s, 6H), 3.22 (t, J=6.6 Hz, 2H), 1.80-2.08 (m, 10H), 1.59-1.77 (m, 2H). ¹³C NMR (125 MHz, CDCl₃, 25° C.) 166.3, 166.2, 163.2, 149.4, 149.2, 125.9, 121.3, 118.5, 118.3, 102.0, 70.8, 70.2, 59.0, 58.9, 58.8, 43.1, 40.6, 38.1, 33.3, 29.7, 28.6, 28.4, 28.3, 6.6. HRMS (ESI+): calcd. (M+H⁺) 709.2098. found 709.2138.

Compound (3). Compound (10) (10 mg, 0.014 mmol) and PPh₃ (8 mg, 0.028 mmol) were dissolved in dry CH₃CN (5 mL) and heated at 60° C., for 3 d. The solvent was removed under a reduced pressure and the obtained blue solid was purified by silica chromatography using CH₃OH in CH₂Cl₂ (gradient 0-20%) and then washed several times with hexanes. The yield was 8.0 mg (62%). ¹H NMR (300 MHz, CDCl₃, 25° C.) 9.39 (t, 1H), 9.23 (t, 1H), 8.07 (s, 1H), 7.94 (s, 1H), 7.70-7.89 (m, 15H), 4.23 (m, 4H), 3.78-3.87 (m, 2H), 3.41-3.61 (m, 10H), 3.37 (s, 3H), 3.30 (s, 6H), 1.78-2.04 (m, 12H). ¹³C NMR (125 MHz, CDCl₃, 25° C.) 165.9, 162.8, 149.0, 148.8, 135.3, 133.9, 133.8, 130.8, 130.7, 125.5, 125.4, 120.9, 118.5, 118.1, 117.8, 101.6, 70.7, 70.1, 58.9, 58.7, 42.7, 40.5, 37.9, 29.6, 29.0, 28.2, 28.1, 22.6. HRMS (ESI+): calcd. (M−I⁻) 843.3886. found 843.3894.

### Example 3

### Synthetic Scheme for Compound (4)

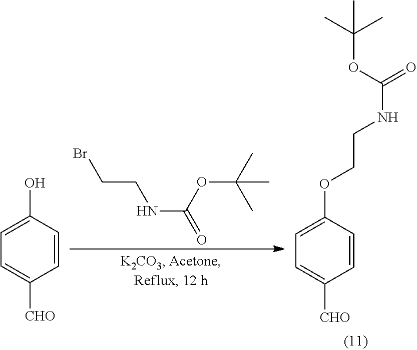 [SMILES: O=Cc1ccc(O)cc1 ; CC(C)(C)OC(=O)NCCBr ; CC(C)(C)OC(=O)NCCOc1ccc(C=O)cc1]

Compound (11). 4-Hydroxybenzaldehyde (1.0 g, 8.19 mmol), 2-t-BOC-aminoethyl-bromide (1.52 g, 6.87 mmol) and potassium carbonate (1.9 g, 13.75 mmol) were refluxed in acetone (20 mL). After 2 h the solution was cooled to rt and water (15 mL) was added. The aqueous solution was extracted with EtOAc (3×30 mL). The combined organic phases were dried over anhydrous Na₂SO₄, concentrated in vacuo, and purified by flash chromatography on silica using CH₂Cl₂ as eluent, affording 1.34 g (62%) product (11). ¹H NMR (300 MHz, CDCl₃, 25° C.) 9.88 (s, 1H), 7.83 (d, J=8.4 Hz, 2H), 6.69 (d, J=9.0 Hz, 2H), 4.98 (s, broad, 1H), 4.10 (t, J=4.8 Hz, 2H), 3.59 (m, 2H), 1.44 (s, 9H). ¹³C NMR (125 MHz, CDCl₃, 25° C.) 190.9, 163.7, 156.0, 132.0, 130.1, 114.8, 79.6, 67.5, 39.9, 28.4.

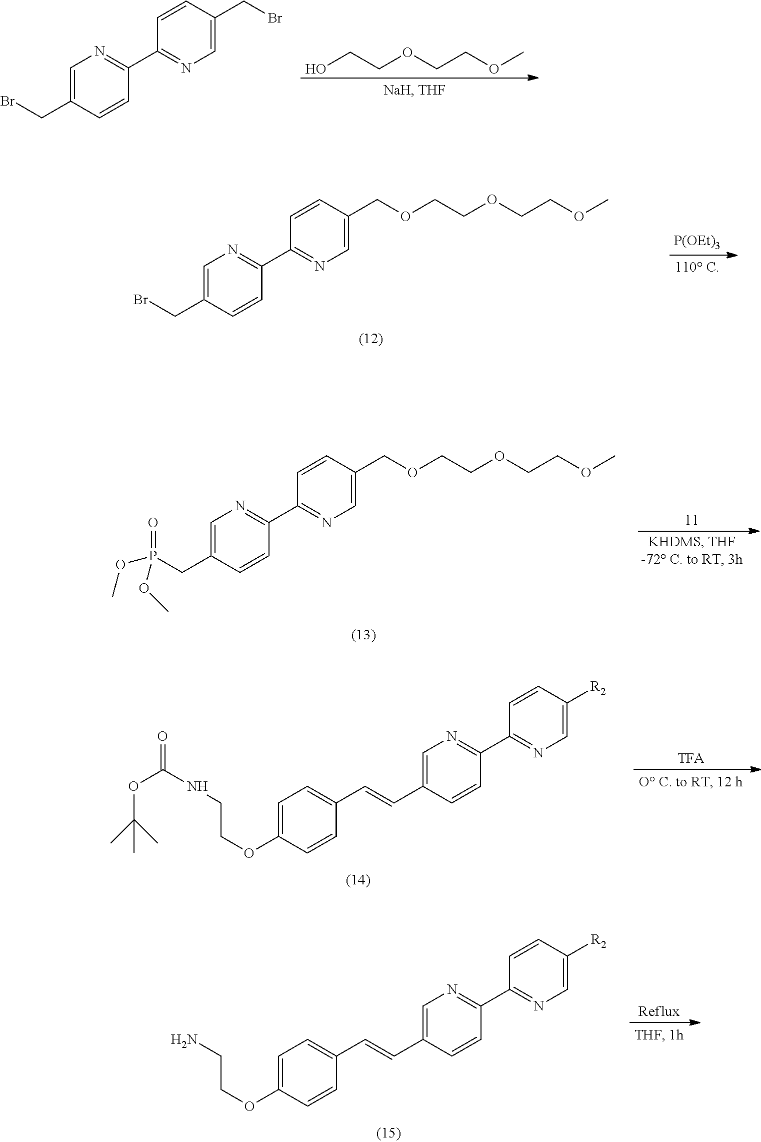

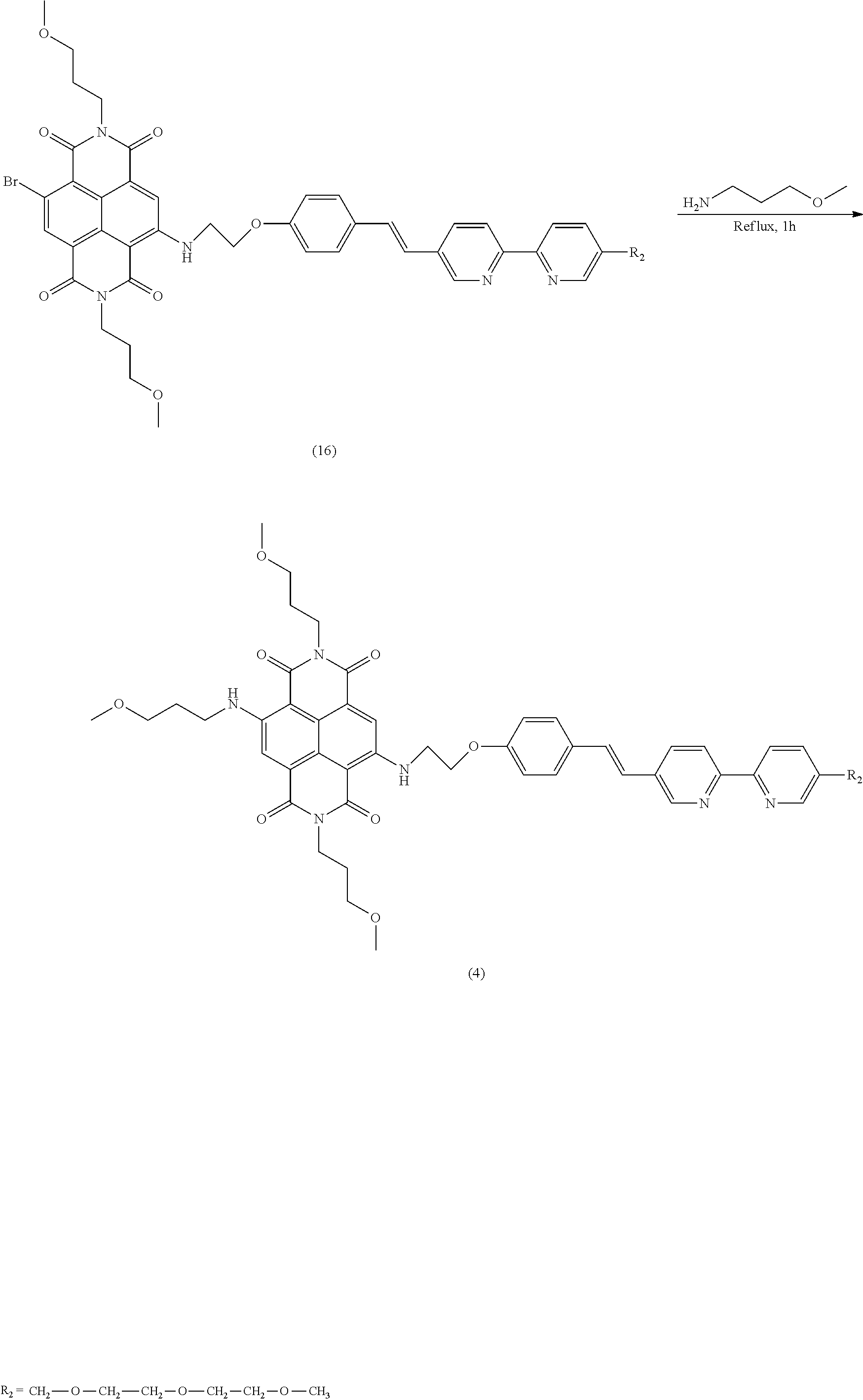

Compound (12).

Sodium hydride, (108 mg, 60% dispersion in mineral oil; 2.71 mmol) was suspended in dry THF (8.0 mL) in a flame-dried flask and it was cooled to 0° C. Di(ethylene glycol)methyl ether (360 mg, 3.0 mmol) was added slowly and stirred under an argon atmosphere for 30 min. The solution was slowly added to 5,5′-bis(bromomethyl)-2,2′-dipyridyl (1.0 g, 5.4 mmol) in THF (50 mL) and stirred for 2 h. See Zhang, L.; Clark, R. J.; Zhu, L. Chem. Eur. J. 2008, 14, 2894-2903. Then most of the THF was removed under vacuum. The residue was partitioned between CH₂Cl₂ and brine. The organic layer was dried over anhydrous Na₂SO₄, filtered and concentrated. The crude product was purified using silica chromatography, using THF in CH₂Cl₂ (gradient 0-10%). The yield of (12) was 1.1 g (53%). ¹H NMR (300 MHz, CDCl₃, 25° C.) 8.68 (s, 1H), 8.63 (s, 1H), 8.38 (d, J=8.1 Hz, 2H), 7.82-7.86 (m, 2H), 4.65 (s, 2H), 4.53 (s, 2H), 3.65-3.54 (m, 8H), 3.39 (s, 3H). ¹³C NMR (125 MHz, CDCl₃, 25° C.) 155.8, 154.9, 149.3, 148.6, 137.6, 136.6, 134.1, 133.6, 121.1, 121.0, 72.0, 70.7, 70.6, 69.9, 59.1, 29.7.

Compound (13).

Compound (12) (700 mg, 1.83 mmol) was dissolved in (EtO)₃P (2.0 mL). The mixture was heated at 110° C. for 4 h. Excess of (EtO)₃P was removed under high vacuum in a fume hood. The residue was partitioned between EtOAc and NaHCO₃ (0.1 M). The organic layer was washed with NaHCO₃ (0.1 M) twice before being dried over anhydrous Na₂SO₄. The solvent was removed under a reduced pressure to afford analytically pure product. Yield: 657 mg (82%). ¹H NMR (300 MHz, CDCl₃, 25° C.) 8.63 (s, 1H), 8.56 (s, 1H), 8.34 (d, J=7.8 Hz, 2H), 7.75-7.84 (m, 2H), 4.64 (s, 2H), 4.06 (q, 4H), 3.55-3.70 (m, 8H), 3.88 (s, 3H), 3.19 (d, J=22 Hz, 2H), 1.27 (t, J=7.2 Hz, 6H). ¹³C NMR (125 MHz, CDCl₃, 25° C.) 155.3, 154.6, 149.9, 148.6, 138.1, 136.4, 133.8, 128.0, 125.5, 120.7, 71.9, 70.6, 69.8, 62.4, 59.0, 31.5, 30.3, 16.4. HRMS (FAB) m/z: calcd (M+Na⁺) 461.1817. found 461.1824.

Compound (14).

In a flame dried flask compounds (13) (300 mg, 0.68 mmol) and (11) (180 mg, 0.68 mmol) were dissolved in dry THF (20 mL) and cooled to −78° C. Potassium bis(trimethylsilyl)amide (1.5 mL, 0.5 M in toluene, 0.75 mmol) was added dropwise. Upon completing the addition, the stirring was continued for 3 h while the temperature rose to rt. The reaction mixture was then partitioned between CH₂Cl₂ and water. The aqueous layer was washed with CH₂Cl₂ (50 mL×3) and the organic portions were combined. The organic portions were dried over Na₂SO₄ followed by solvent removal under vacuum. The compound was isolated via silica chromatography using 0-25% THF in CH₂Cl₂. The isolated yield of (14) was 160 mg (43%). ¹H NMR (300 MHz, CDCl₃, 25° C.) 8.73 (s, 1H), 8.64 (s, 1H), 8.37 (d, J=8.7 Hz, 2H), 7.95 (d, J=7.8 Hz, 1H), 7.83 (d, J=7.5 Hz, 1H), 7.49 (d, J=8.4 Hz, 2H), 7.18 (d, J=16.8 Hz, 1H), 7.00 (d, J=16.2 Hz, 1H), 6.91 (d, J=8.7 Hz, 2H), 5.01 (s, broad, 1H), 4.65 (s, 2H), 4.05 (t, J=4.8 Hz, 2H), 3.75-3.54 (m, 10H), 3.39 (s, 3H), 1.46 (s, 9H). ¹³C NMR (125 MHz, CDCl₃, 25° C.) 158.8, 155.9, 155.4, 154.4, 148.7, 148.0, 136.5, 133.6, 133.3, 133.2, 130.4, 129.9, 128.1, 122.8, 121.0, 120.7, 114.8, 79.6, 72.0, 70.7, 69.8, 67.3, 59.1, 40.1, 28.4. HRMS (FAB) m/z: calcd (M+Na⁺) 572.2737. found 572.2746.

Compound (15).

Compound (14) (100 mg, 0.18 mmol) dissolved in CH₂Cl₂ (2.0 mL) was slowly added to trifluoroacetic acid (5.0 mL) at 0° C. The reaction mixture was stirred at rt for 12 h. Then the reaction mixture was diluted with CH₂Cl₂ (50 mL) followed by extraction with a NaOH solution (0.5 M, saturated with NaCl) three times. The organic layer was dried over K₂CO₃ before the solvent was removed under vacuum to afford analytically pure product (15). Yield: 75 mg (92%). ¹H NMR (300 MHz, CDCl₃, 25° C.) 8.73 (s, 1H), 8.63 (s, 1H), 8.37 (d, J=8.1 Hz, 2H), 7.94 (d, J=8.4 Hz, 1H), 7.82 (d, J=8.4 Hz, 1H), 7.48 (d, J=8.4 Hz, 2H), 7.18 (d, J=16.2 Hz, 1H), 6.99 (d, J=16.2 Hz, 1H), 6.92 (d, J=9.0 Hz, 2H), 4.65 (s, 2H), 4.02 (t, J=4.8 Hz, 2H), 3.55-3.71 (m, 8H), 3.39 (s, 3H), 3.10 (t, J=4.8 Hz, 2H). ¹³C NMR (125 MHz, CDCl₃, 25° C.) 159.2, 155.5, 154.4, 148.7, 147.9, 136.5, 133.6, 133.4, 133.1, 130.5, 129.7, 128.1, 122.6, 121.0, 120.7, 114.8, 72.0, 70.7, 70.3, 69.8, 59.1, 41.6. HRMS (FAB) m/z: calcd (M+Na⁺) 472.2212. found 472.2220.

Compound (16).

Compound (7) (63 mg, 0.11 mmol) and (15) (50 mg, 0.11 mmol) were refluxed in THF for 1 h. The solvent was removed under a reduced pressure and the obtained red solid was purified by silica chromatography using THF in CH₂Cl₂ (gradient 0-10%). The yield of (16) was 44 mg (42%). ¹H NMR (300 MHz, CDCl₃, 25° C.) 10.43 (t, 1H), 8.87 (s, 1H), 8.72 (s, 1H), 8.63 (s, 1H), 8.45 (s, 1H), 8.37 (d, J=7.8 Hz, 2H), 7.94 (d, J=6.6 Hz, 1H), 7.82 (d, J=6.9 Hz, 1H), 7.48 (d, J=8.4 Hz, 2H), 7.17 (d, J=16.8 Hz, 1H), 6.99 (d, J=16.8, 1H), 6.95 (d, J=8.4 Hz, 2H), 4.65 (s, 2H), 4.27-4.38 (m, 6H), 4.04 (q, 2H), 3.70-3.65 (m, 6H), 3.58-3.49 (m, 6H), 3.39 (s, 3H), 3.33 (s, 6H), 2.03 (m, 4H). ¹³C NMR (125 MHz, CDCl₃, 25° C.) 165.9, 162.0, 161.9, 161.5, 158.3, 155.4, 154.5, 152.0, 148.7, 148.0, 138.4, 136.5, 133.7, 133.2, 130.3, 130.2, 128.6, 128.1, 127.4, 123.6, 123.4, 123.0, 121.7, 121.0, 120.7, 120.5, 115.0, 100.7, 72.0, 70.7, 70.6, 69.9, 66.7, 59.1, 58.7, 58.6, 42.6, 39.1, 38.1, 28.1, 28.0. HRMS (ESI) m/z: calcd (M+H⁺) 936.2819. found 936.2793.

Compound (4).

Compound (16) (25 mg, 0.026 mmol) and 3-methoxypropylamine (2.0 mL) were refluxed under an argon atmosphere for 1 h. Then the reaction mixture was diluted with CH₂Cl₂ (50 mL) followed by extraction with a dilute HCl solution (0.5 M, saturated with NaCl) three times. The organic layer was dried over K₂CO₃ before the solvent was removed under a reduced pressure and the obtained blue solid was purified via silica chromatography using THF in CH₂Cl₂ (gradient 0-30%). Yield of (4) was 15 mg (62%). ¹H NMR (300 MHz, CDCl₃, 25° C.) 9.66 (t, 1H), 9.45 (t, 1H), 8.72 (s, 1H), 8.63 (s, 1H), 8.36 (d, J=9.0 Hz, 2H) 8.20 (s, 1H), 8.12 (s, 1H), 7.93 (d, J=8.4 Hz, 1H), 7.82 (d, J=8.4 Hz, 1H), 7.48 (d, J=9 Hz, 2H), 7.16 (d, J=16.8 Hz, 1H), 6.95 (d, J=16.8 Hz, 1H), 6.97 (d, J=8.4 Hz, 2H), 4.64 (s, 2H), 4.28-4.33 (m, 6H), 3.94 (q, 2H), 3.49-3.71 (m, 16H), 3.39 (s, 6H), 3.35 (s, 6H), 2.02 (m, 6H). ¹³C NMR (125 MHz, CDCl₃, 25° C.) 166.2, 166.0, 163.0, 158.6, 155.4, 149.4, 148.9, 148.7, 148.0, 136.5, 133.6, 133.3, 133.1, 130.4, 130.1, 128.1, 125.8, 125.6, 122.8, 121.4, 121.1, 121.0, 120.7, 118.6, 118.0, 115.0, 102.6, 101.7, 72.0, 70.7 70.6, 70.0, 69.8, 66.7, 59.1, 58.9, 58.6, 42.5, 40.4, 37.9, 29.5, 28.2. HRMS (ESI) m/z: calcd (M+H⁺) calcd 945.4398. found 945.4387.

### Example 4

### Synthetic Scheme for Compound (5)

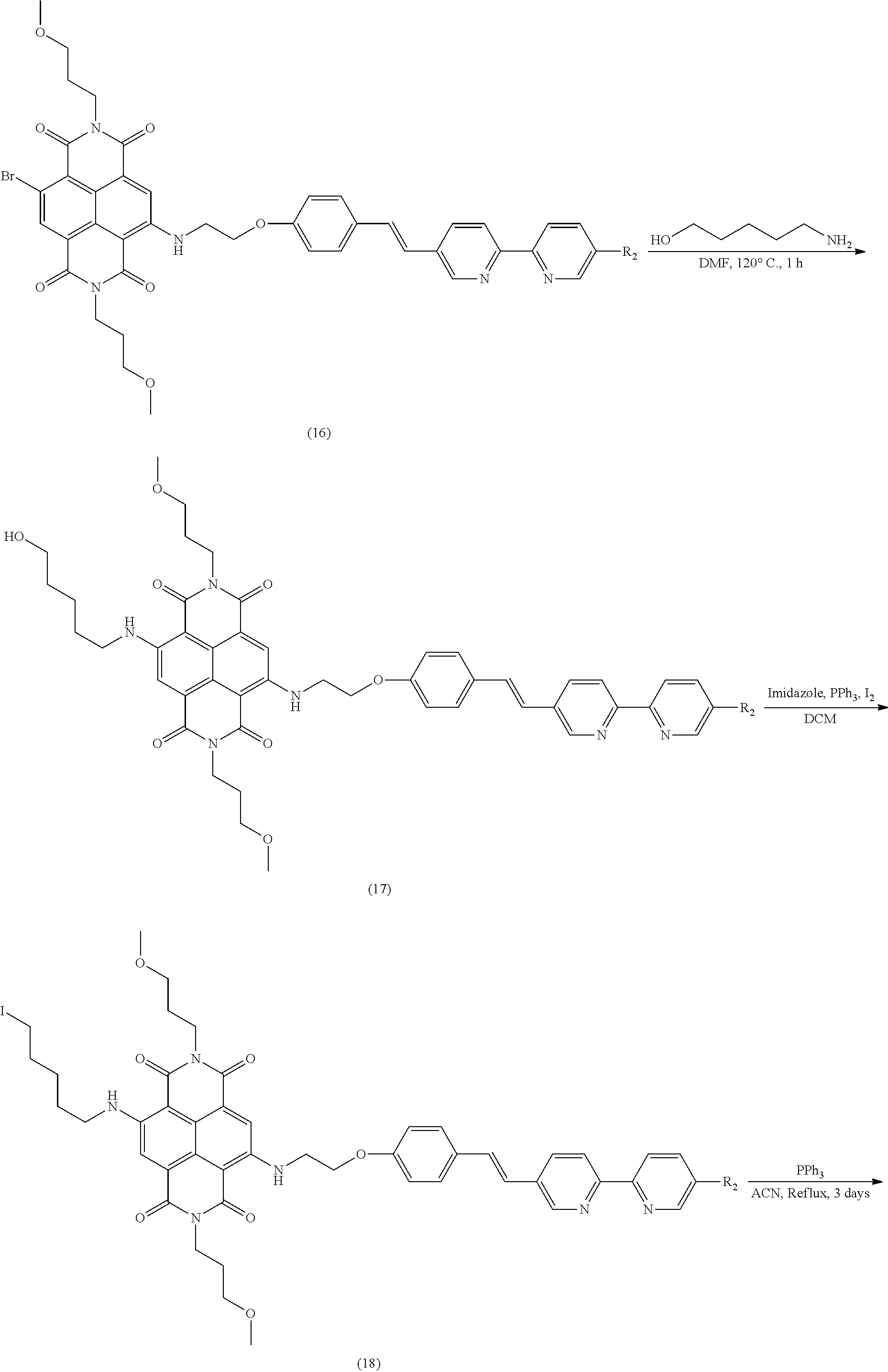

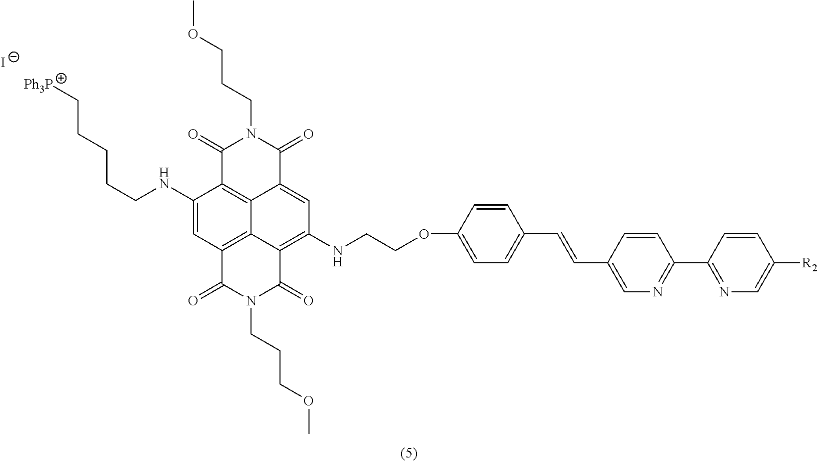

Compound (17). Compound (16) (20 mg, 0.02 mmol) and 5-aminopentanol (20 mg, 0.2 mmol) were dissolved in DMF (2.0 mL) and heated at 110° C. under an argon atmosphere for 1 h. The solvent was removed under a reduced pressure. The blue residue obtained was dissolved in CH₂Cl₂ (50 mL) followed by extraction with a dilute HCl solution (0.5 M, saturated with NaCl) three times. The organic layer was dried over anhydrous Na₂SO₄ before the solvent was removed under a reduced pressure and the obtained blue solid was purified by silica chromatography using THF in CH₂Cl₂ (gradient 0-50%). The yield of (17) was 11 mg (53%). ¹H NMR (300 MHz, CDCl₃, 25° C.) 9.66 (t, 1H), 9.38 (t, 1H), 8.72 (s, 1H), 8.63 (s, 1H), 8.37 (d, J=8.4 Hz, 2H), 8.21 (s, 1H), 8.09 (s, 1H), 7.93 (d, J=10.8 Hz, 1H), 7.81 (d, J=10.8 Hz, 1H), 7.47 (d, J=9.0 Hz, 2H), 7.17 (d, J=16.2 Hz, 1H), 7.00 (d, J=16.2 Hz, 1H), 6.96 (d, J=9.0 Hz, 2H), 4.64 (s, 2H), 4.25-4.34 (m, 6H), 3.94 (q, 2H), 3.57-3.71 (m, 8H), 3.48-3.57 (m, 8H), 3.39 (s, 3H), 3.34 (s, 6H), 2.02 (m, 4H), 1.86 (q, 2H), 1.61-1.70 (m, 4H). ¹³C NMR (125 MHz, CDCl₃, 25° C.) 166.3, 163.2, 158.8, 155.6, 154.6, 149.4, 149.1, 148.8, 148.1, 136.7, 133.8, 133.5, 133.3, 130.5, 130.3, 128.2, 126.0, 125.8, 123.0, 121.5, 121.2, 121.1, 120.9, 118.6, 118.2, 115.2, 102.7, 101.7, 72.2, 70.9, 70.8, 70.0, 66.9, 62.8, 59.3, 58.8, 43.2, 42.6, 38.1, 32.5, 29.3, 28.4, 23.6. HRMS (ESI) m/z: calcd (M+H⁺) 959.4554. found 959.4554.

Compound (18).

PPh₃ (6.0 mg, 0.02 mmol) and imidazole (2.0 mg, 0.02 mmol) were dissolved in dry CH₂Cl₂ (4.0 mL) and cooled to 0° C. Iodine (5.0 mg, 0.02 mmol) was added and stirred for 10 min. Compound (17) (10 mg, 0.01 mmol) dissolved in dry CH₂Cl₂ was added dropwise and stirred for 12 h at rt overnight. The solvent was removed under a reduced pressure and the obtained blue solid was purified by silica chromatography using THF in CH₂Cl₂ (gradient 0-30%). The yield of 18 was 6.0 mg (54%). ¹H NMR (300 MHz, CDCl₃, 25° C.) 9.68 (t, 1H), 9.39 (t, 1H), 8.73 (s, 1H), 8.67 (s, 1H), 8.37 (d, J=8.4 Hz, 2H), 8.22 (s, 1H), 8.09 (s, 1H), 8.01 (d, J=10.8 Hz, 1H), 7.81 (d, J=10.8 Hz, 1H), 7.48 (d, J=8.4 Hz, 2H), 7.17 (d, J=16.2 Hz, 1H), 6.96 (m, 3H), 4.65 (s, 2H), 4.25-4.34 (m, 6H), 3.93 (m, 2H), 3.61-3.73 (m, 6H), 3.46-3.59 (m, 8H), 3.38 (s, 3H), 3.35 (s, 6H), 3.23 (t, J=6.6 Hz, 2H), 1.86-2.00 (m, 6H), 1.72-1.58 (m, 4H). HRMS (ESI) m/z: calcd (M+H⁺) calcd 1069.3572. found 1069.3562.

Compound (5).

Compound (18) (6.0 mg, 0.006 mmol) and PPh₃ (3.0 mg, 0.012 mmol) were dissolved in dry CH₃CN (4.0 mL) and heated at 60° C. for 3 d. The solvent was removed under a reduced pressure and the obtained blue solid was purified via silica chromatography using CH₃OH in CH₂Cl₂ (gradient 0-30%). The yield of product was 4.0 mg (59%). ¹H NMR (500 MHz, CDCl₃, 25° C.) 9.67 (t, 1H), 9.37 (t, 1H), 8.72 (s, 1H), 8.63 (s, 1H), 8.37 (d, J=8.4 Hz, 2H), 8.35 (s, 1H), 8.22 (s, 1H), 7.94 (d, J=10.8 Hz, 1H), 7.93-7.60 (m, 16H), 7.47 (d, J=8.4 Hz, 2H), 7.16 (d, J=16.2 Hz, 1H), 7.02 (d, J=16.2 Hz, 1H), 6.97 (d, J=9.0 Hz, 2H), 4.64 (s, 2H), 4.24-4.33 (m, 8H), 3.95 (m, 2H), 3.62-3.73 (m, 6H), 3.58 (m, 8H), 3.38 (s, 3H), 3.34 (s, 6H), 1.86-2.00 (m, 6H), 1.58-1.72 (m, 4H). HRMS (ESI) m/z: calcd (M−I⁻) 1203.5360. found 1203.5312.

### Example 6

### V. Fluorescence Microscopy

The localization properties of (2-5) were determined via costaining experiments using mCerulean3 TOMM-20, a mitochondrial-specific fusion of the cyan fluorescent protein (CFP) mCerulean3. mCerulean3 is a bright monomeric FP with an emission maximum of 475 nm. HeLa S3 cells were seeded onto Bioptechs Delta-T dishes 48 h prior to experimentation, then transfected about 24 h prior to loading the indicator using an Effectene Transfection Reagent Kit (Qiagen) and 1 μg of DNA. A 1:1 mixture of Dulbecco's modified Eagle's medium and Ham's F-12 (Invitrogen) supplemented with 12.5% fetal bovine serum was used as the culture medium. On the day of experimentation cells were rinsed twice with media and incubated with indicator 5 (1.8 μM) for 30 min. Prior to imaging cells were again rinsed twice with media, then provided with 1 mL of fresh media.

All cells were imaged using an Olympus Fluoview FV1000 confocal laser scanning microscope with an Olympus PLAPO 60× oil-immersion objective (NA=1.4). During experimentation cells were maintained at 37° C. and 5.0% CO₂ using a Bioptechs Delta T4 Culture Dish Controller. Individual cells were imaged sequentially in two channels, using a 543 nm Helium-Neon laser line to excite the indicator and a 405 nm diode laser line to excite the FP. Cells were selected by the quality of FP expression, as the indicators tended to load homogenously into the cells. All data was collected using FluoView Software (Olympus). Laser power and detection settings were optimized for each image to fill LUTs. A pinhole size of 200 μm was used for each image. Merged-channel images were produced using Elements software (Nikon).

For performing zinc(II) supplementation experiments, HeLa S3 cells were seeded onto Bioptechs Delta-T dishes approximately 24 h prior to the addition of ZnCl₂. The culture medium is the same as used for the colocalization experiments (1:1 DMEM to Ham's F12 with Fetal Bovine Serum). Cells were rinsed twice with media and incubated with fluorescent indicator 5 (1.8 μM) for 30 min. The dish was again rinsed twice with media and incubated an additional 10 min in 1 mL of media, with solutions containing either 50 μM ZnCl₂ and 5 μM sodium pyrithione or 0 μM ZnCl₂ and 5 μM sodium pyrithione. For each experiment, laser power and detection settings were optimized to fill LUTs for the first cell imaged in the presence of 50 μM ZnCl₂, with the same settings being used for each subsequent image and for cells imaged without supplemental ZnCl₂.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A compound capable of measuring the concentration of zinc(II) ion in a composition, the compound comprising a covalently-linked assembly of a fluorescence resonance energy transfer donor chromophore and a fluorescence resonance energy transfer acceptor chromophore;
wherein the fluorescence resonance energy transfer donor chromophore comprises a functional group capable of chelating zinc(II) ion.

2. The compound of claim 1 further comprising a mitochondrial targeting moiety.

3. The compound of claim 2 wherein the mitochondrial targeting moiety comprises triphenyl-phosphonium.

4. The compound of claim 1 wherein the fluorescence resonance energy transfer donor chromophore is derived from a compound having the following structure: 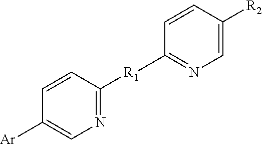
wherein
R₁ is a connecting moiety, which maintains conjugation between the nitrogen atoms;
R₂ is an alkyl group, a hydroxylalkyl group, an alkoxy group, an alkoxyalkyl group, or a poly(alkoxy) group; and
Ar is an aryl moiety containing a functional group capable of reacting with an ether moiety.

5. The compound of claim 1 wherein the fluorescence resonance energy transfer donor chromophore is derived from a compound having the following structure: 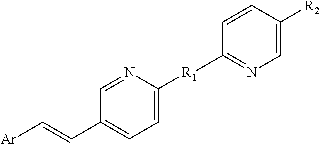
wherein
R₁ is a connecting moiety, which maintains conjugation between the nitrogen atoms;
R₂ is an alkyl group, a hydroxylalkyl group, an alkoxy group, an alkoxyalkyl group, or a poly(alkoxy) group; and
Ar is an aryl moiety containing a functional group capable of reacting with an ether moiety.

6. The compound of claim 1 wherein the fluorescence resonance energy transfer donor chromophore is derived from a compound selected from the group consisting of: 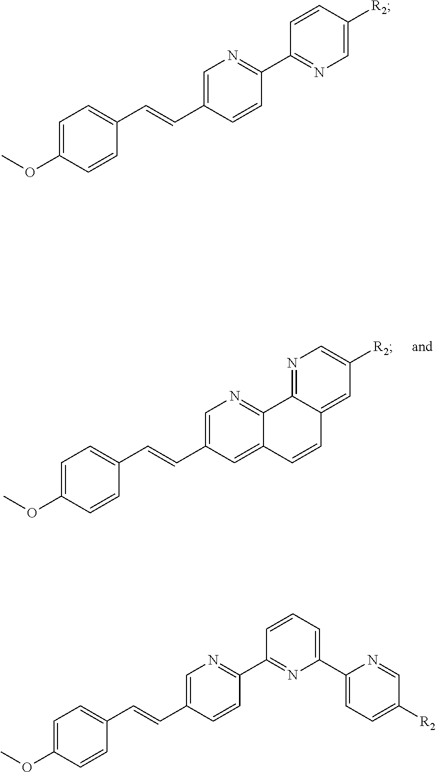
wherein R₂ is an R₂ is an alkyl group, a hydroxylalkyl group, an alkoxy group, an alkoxyalkyl group, or a poly(alkoxy) group.

7. The compound of claim 1 wherein the fluorescence resonance energy transfer donor chromophore is derived from 5-(4-methoxystyryl)-5′-methyl-2,2′-bipyridine (1) having the following structure:

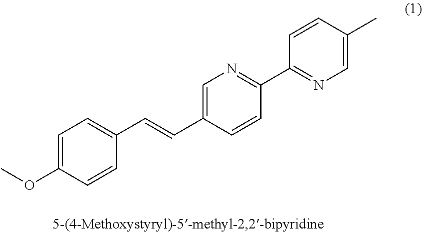

8. The compound of claim 1 wherein the fluorescence resonance energy transfer acceptor chromophore is derived from a naphthalenediimide.

9. The compound of claim 8 wherein the naphthalenediimide has the following general structure: 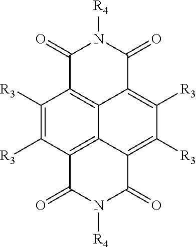
wherein
R₃ are each independently hydrogen, an alkyl group, a hydroxyalkyl group, an alkoxy group, an alkylalkoxy group, a poly(alkoxy) group, or an alkylamino group; and
R₄ are each independently hydrogen, an alkyl group, a hydroxyalkyl group, an alkoxy group, an alkylalkoxy group, a poly(alkoxy) group, an alkylamino group, an aryl moiety, or propargyl.

10. The compound of claim 9 wherein two R₃ are alkylamino groups.

11. The compound of claim 9 wherein at least one of the R₄ groups comprises methoxypropyl.

12. The compound of claim 8 wherein the naphthalenediimide has the following general structure: 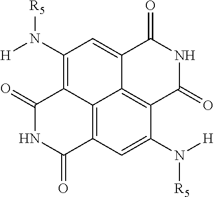
wherein each R₅ is independently hydrogen, an alkyl, a hydroxyalkyl, or an alkoxy.

13. The compound of claim 12 wherein R₅ comprises a lipophilic mitochondrial targeting moiety.

14. The compound of claim 8 wherein the naphthalenediimide has the following structure: 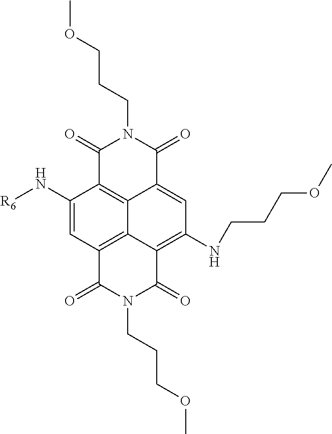
wherein R₆═CH₂—CH₂—CH₂—O—CH₃ or CH₂—CH₂—CH₂—CH₂—CH₂—PPh₃I

15. The compound of claim 1 wherein the fluorescence resonance energy transfer acceptor chromophore is derived from the following structure:

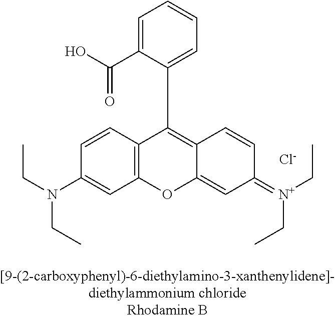 [SMILES: CCN(CC)c1ccc2c(-c3ccccc3C(=O)O)c3ccc(=[N+](CC)CC)cc-3oc2c1 ; [Cl-]]

16. The compound of claim 1 wherein the fluorescence resonance energy transfer acceptor chromophore is derived from the following structure:

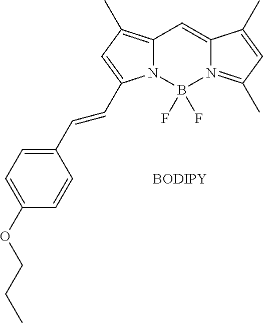

17. A compound having the structure: 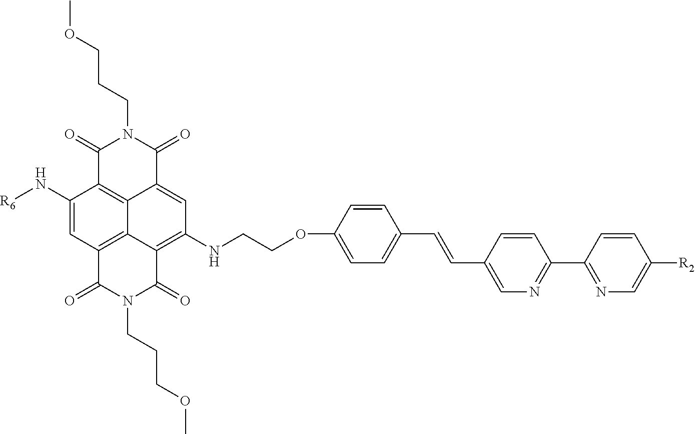
wherein:
R₆ is CH₂—CH₂—CH₂—O—CH₃ or CH₂—CH₂—CH₂—CH₂—CH₂—PPh₃I; and
R₂ is CH₂—O—CH₂—CH₂—O—CH₂—CH₂—O—CH₃ or CH₂—O—CH₂—CH₂—O—CH₂—CH₂—O—CH₃.

18. A compound having the structure:

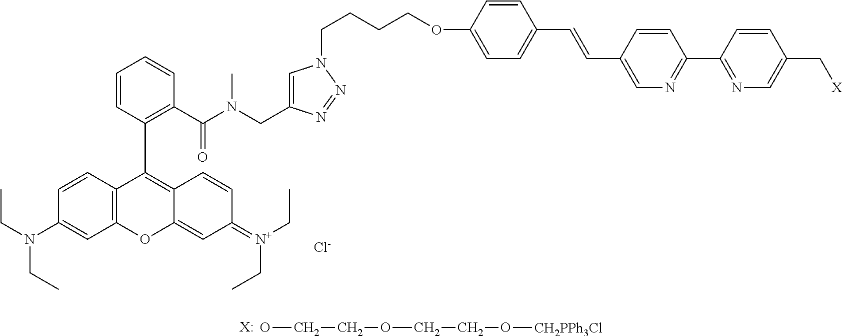

19. A compound having the structure:

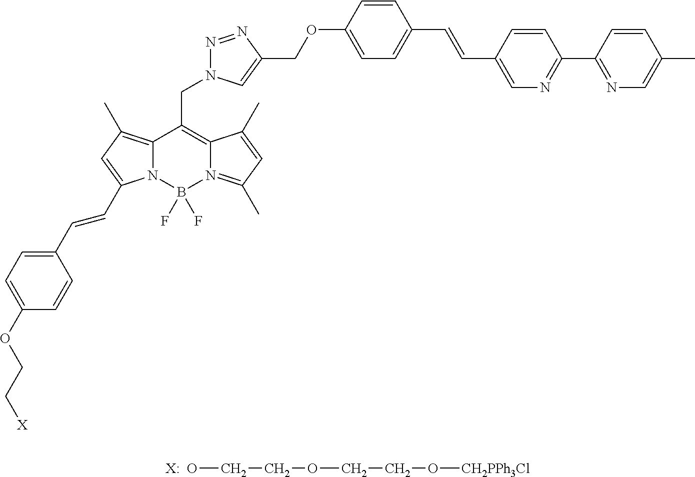

20. A method of determining the zinc(II) ion concentration in a composition comprising zinc(II) ions, the method comprising:
contacting the composition comprising zinc(II) ions with a compound comprising a covalently-linked assembly of a fluorescence resonance energy transfer donor chromophore and a fluorescence resonance energy transfer acceptor chromophore, wherein the fluorescence resonance energy transfer donor chromophore comprises a functional group capable of chelating zinc(II) ion, and wherein said contact causes the fluorescence resonance energy transfer donor chromophore to chelate zinc(II) ion; and
irradiating the composition to thereby excite the zinc(II) ion chelated fluorescence resonance energy transfer donor chromophore, which subsequently transfers the excitation energy (non-radiatively) to the fluorescence resonance energy transfer acceptor chromophore, and allows for the occurrence of emission from the acceptor chromophore; and
detecting fluorescent emission from the fluorescence resonance energy transfer acceptor chromophore.

21. The method of claim 20 wherein the compound has the structure: 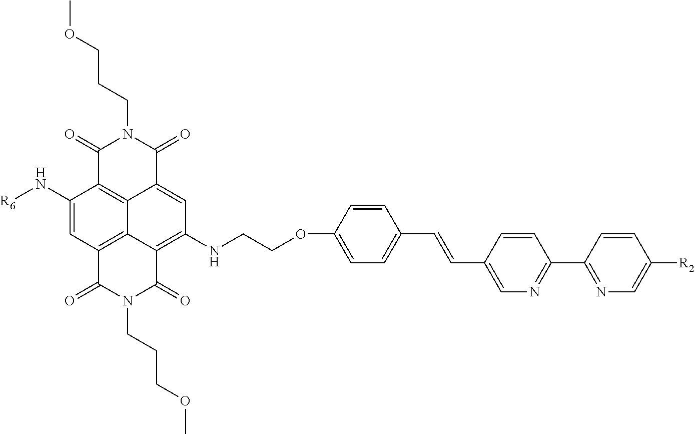
wherein:
R₆ is CH₂—CH₂—CH₂—O—CH₃ or CH₂—CH₂—CH₂—CH₂—CH₂—PPh₃I; and
R₂ is CH₂—O—CH₂—CH₂—O—CH₂—CH₂—O—CH₃ or CH₂—O—CH₂—CH₂—O—CH₂—CH₂—O—CH₃.

22. The method of claim 20 wherein the compound has the structure:

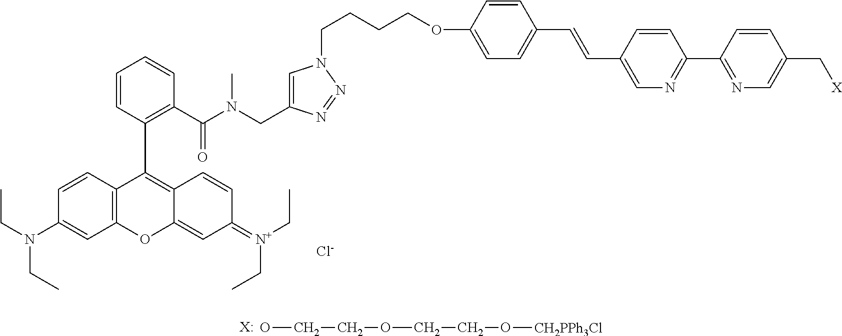

23. The method of claim 20 wherein the compound has the structure: [chemical structure]
